SEQUENCE LISTING (2 sequences)

SEQ ID NO: 1 - DNA, 27 bp, synthetic construct
  source 1..27: mol_type = other DNA
tgctatgtcagttccccttggttctct

SEQ ID NO: 2 - DNA, 30 bp, synthetic construct
  source 1..30: mol_type = other DNA
agttggaggacatcaagcagccatgcaaat

(19) United States
(12) Patent Application Publication
(10) Pub. No.: US 2025/0195415 A1
(43) Pub. Date: Jun. 19, 2025

(54) BIODEGRADABLE CONTROLLED RELEASE ANTIVIRAL AGENT IMPLANTS

(71) Applicant: Yale University, New Haven, CT (US)

(72) Inventors: W. Mark Saltzman, New Haven, CT (US); Jagadish Beloor, New Haven, CT (US); Shalley N. Kudalkar, New Haven, CT (US); Karen S. Anderson, New Haven, CT (US); Fan Yang, New Haven, CT (US); Gina Buzzelli, New Haven, CT (US); Jinal Pothupitiya, New Haven, CT (US); Priti Kumar, New Haven, CT (US)

(21) Appl. No.: 18/849,428

(22) PCT Filed: Mar. 21, 2023
(86) PCT No.: PCT/US2023/064762

§ 371 Date: Sep. 20, 2024

Related U.S. Application Data
(60) Provisional application No. 63/321,934, filed on Mar. 21, 2022.

(51) Int. Cl. A61K 9/00; A61K 9/50; A61K 31/513; A61K 31/52; A61P 31/14

(52) U.S. Cl. CPC A61K 9/0024; A61K 9/5031; A61K 31/513; A61K 31/52; A61P 31/14

(57) ABSTRACT

Biodegradable controlled release agent implants and methods of making and using thereof, are preferably formed of poly(ω-pentadecalactone-co-p-dioxanone) [poly(PDL-co-DO)] or poly(ethylene brassylate-co-dioxanone), a family of polyester copolymers that degrade slowly in the presence of water. The material is suitable as the basis of a biodegradable controlled agent release implant that provides sustained release of a therapeutic such as an antiviral, thereby significantly increasing patent compliance and efficacy. Results show long term co-administration of agents having greater than additive efficacy. The implant may be inserted subcutaneously, allowing degradation over a period of up to about 18 or 24 months, eliminating the need for removal by a trained practitioner.

### CROSS-REFERENCE TO RELATED APPLICATIONS

This application claims benefit of and priority to U.S. Ser. No. 63/321,934 filed Mar. 21, 2022, which is incorporated herein by reference in its entirety.

### STATEMENT REGARDING FEDERALLY SPONSORED RESEARCH

This invention was made with government support under AI044616, AI155072, GM032136, and GM049551 awarded by National Institutes of Health. The government has certain rights in the invention.

### REFERENCE TO THE SEQUENCE LISTING

The Sequence Listing submitted as a text file named “YU_8099_PCT_ST26.xml” created on Mar. 21, 2023, and having a size of 2,864 bytes is hereby incorporated by reference pursuant to 37 C.F.R. § 1.834 (c) (1).

### FIELD OF THE INVENTION

This invention is in the field of controlled agent release implants, specifically biodegradable controlled agent release implants.

### BACKGROUND OF THE INVENTION

An estimated 38 million people are living with HIV infection Worldwide. In the United States, approximately 1.2 million people aged 13 years and older are living with HIV infection, and almost 1 in 7 (14%) are unaware of their infection. The estimated incidence of HIV in the United States has declined by 7% between 2014 and 2018, and is now at about 36,400 new infections occurring each year. Significant advances in antiretroviral therapy have been made since the introduction of zidovudine (AZT) in 1987.

With the advent of highly active antiretroviral therapy (HAART), HIV-1 infection is now manageable as a chronic disease in patients who have access to medication and who achieve durable virologic suppression. Excess mortality among patients with AIDS was nearly halved in the HAART era but remains approximately 5 times higher in patients with AIDS than in HIV-infected patients without AIDS. Risk factors for excess mortality include a viral load greater than 400 copies/mL, CD4 count less than 200 cells/mL, and cytomegalovirus retinitis. Despite reductions in mortality with antiretroviral therapy, overall mortality remains 6 times higher in persons with HIV than the general population.

The HIV pandemic continues to affect over 37 million people worldwide and nearly half are on antiretroviral (ARV) therapy. A major therapeutic challenge in treatment and prevention of HIV-I is non-adherence due to daily dosing of HIV agents. Hence, long-acting (LA) ARV formulations are urgent to tackle this adherence problem. However, LA-ARV formulations, which are currently approved or are in clinical trials, require large and repetitive doses and cannot be removed in case of adverse events after administration.

HAART provides effective treatment options for treatment-naive and treatment-experienced patients. Pharmacologic agent classes include: Nucleoside reverse transcriptase inhibitors (NRTIs), Non-nucleoside reverse transcriptase inhibitors (NNRTIs), Protease inhibitors (PIs), Integrase inhibitors (INSTIs), Fusion inhibitors (FIs), Chemokine receptor antagonists (CCR5 antagonists), and Entry inhibitors (CD4-directed post-attachment inhibitors).

Antiretroviral therapy (ART) has revolutionized the treatment of HIV-AIDS and the disease is no longer a death sentence. However, as ART does not cure, treatment must be life-long. Currently, there is no available oral ART agent or formulation with a dosing frequency of less than once daily. Hence, non-adherence to daily oral medication remains the most significant barrier to achieve long-term suppression of HIV replication and prevention of the emergence of agent-resistant virus. Numerous studies show direct correlation between ART adherence and reduction in viral loads and elevation in CD4 T cell counts. Studies also show that the side-effects and psychological reactions to taking ART leads to non-adherence in HIV-positive patients, particularly younger individuals, who account for more than half of all new HIV infections. Thus, new agents with improved pharmacological properties, and a long-acting antiretroviral agent delivery system that could reduce the dosing frequency to weekly, monthly, or even longer periods of time could represent a significant advance in HIV treatment, especially in high-risk populations.

Antiretroviral therapy is recommended as soon as possible for all individuals with HIV who have detectable viremia. Most patients can start with a 3-agent regimen or now a 2-agent regimen, which includes an integrase strand transfer inhibitor. Effective options are available for patients who may be pregnant, those who have specific clinical conditions, such as kidney, liver, or cardiovascular disease, those who have opportunistic diseases, or those who have health care access issues. Recommended for the first time, a long-acting antiretroviral regimen injected once every 4 weeks for treatment or every 8 weeks pending approval by regulatory bodies and availability. For individuals at risk for HIV, pre-exposure prophylaxis with an oral regimen is recommended or, pending approval by regulatory bodies and availability, with a long-acting injection given every 8 weeks.

The majority of the approved and investigational antiretrovirals are not suitable for long-acting formulations due to insufficient antiviral potency, side effects, or suboptimal physicochemical properties. The long-acting injectable nanoformulation containing the combination of rilpivirine (non-nucleoside reverse transcriptase inhibitor, NNRTI) and cabotegravir (integrase inhibitor) called Cabenuva requires large injection volumes and multiple injections to achieve a pharmacokinetic profile required for a monthly dosing. Cabenuva was rejected by the FDA due to chemical manufacturing and safety concerns in December 2019, although an amended application received approval in late January 2021. Additionally, a critical drawback of an injectable, long-acting nanoformulation is that they cannot be removed from the body in case of a medical emergency and can magnify any adverse side effects due to maintenance of detectable concentrations of agent in the bloodstream for longer periods of time. Some of these limitations can be addressed by developing removable implants, which are biodegradable and can also provide extended agent release over many months. Currently, 4′-Ethynyl-2-fluoro-2′-deoxyadenosine, EFdA (also called MK-8591 and Islatravir) an investigational nucleoside reverse transcriptase inhibitor (NRTI) and dolutegravir an integrase strand transfer inhibitor have been tested as removable, long-acting implants. For EFdA, pharmacokinetic studies evaluating the implants in rodents showed sustained levels of agent for up to 6 months that would be sufficient for viral suppression although antiviral efficacy was not reported. The dolutegravir implants delivered agent up to 9-months and showed some viral suppression and protection after vaginal challenge in animal models however agent resistance and viral breakthrough were noted as early as 19 days post therapy. These studies indicate results for removable HIV agent implants but reveal the limitations of monotherapy and the likely need for a multiagent combination.

The success and long-term experience of HIV therapeutic regimens containing two antiviral agent combinations of an NRTI and NNRTI are well documented, and a number of these agents have shown additive antiviral potency in cell culture.

Patient adherence to lifelong HIV therapeutic regimens is limited by the necessity of daily dosing of medications. Therefore a number of recent research efforts have focused on long-acting, extended release formulations summarized in a recent review (Weld E D, Current opinion in HIV and AIDS 15 (1): 33-41 (2020)). Cabenuva, an extended release formulation requiring monthly injections of each of the two agents in combination, has been shown to be non-inferior in Phase II clinical trials and is currently under consideration for FDA approval (Orkin C, New England Journal of Medicine 382 (12): 1124-1135 (2020)). Further advances have been reported in preclinical studies with AIDS agents formulated as long-acting, extended release implants (Weld E D, Current opinion in HIV and AIDS 15 (1): 33-41 (2020)). These include an in situ generated implant with dolutegravir as a monotherapy for HIV treatment and prevention.

It is therefore an object of the present invention to provide an implantable agent delivery system that passively administers therapeutic doses of a therapeutic such as an antiviral.

It is a further object of the invention to provide an implantable long term sustained agent delivery system that is biodegradable.

It is a further object of the invention to provide methods of inserting the implantable agent delivery system.

It is a further object of the invention to provide methods of making the implantable agent delivery system.

### SUMMARY OF THE INVENTION

A long acting (“LA”) agent delivery device has been developed. In a preferred embodiment, two agents are administered in combination, a non-nucleoside reverse transcriptase inhibitor and a nucleoside reverse transcriptase inhibitor (NNRTI/NRTI). In the most preferred embodiment, these are the computationally designed NNRTI, Compound I, and NRTI, EFdA, delivered in the form of removable implant or PLGA nanoformulations.

Biodegradable polymeric implants and methods of making and using thereof, are provided herein. In a preferred embodiment, the implant has a biocompatible polyester copolymer composition encapsulating an agent for long term sustained release, preferably an antiviral, most preferably an anti-HIV agent(s). In the most preferred embodiment, the polyester is a poly(ω)-pentadecalactone-co-p-dioxanone) [poly(PDL-co-DO)] or a poly(ethylene brassylate-co-dioxanone) (also designated a poly(ethylene brassylate-co-p-dioxanone), a family of degradable polyester copolymers that degrade slowly in the presence of water. The material is semi-crystalline over all copolymer compositions, suitable for controlled delivery of molecules, retention of mechanical properties during degradation, and biocompatible. Hence, the material is suitable as the basis of a biodegradable controlled agent release implant that provides sustained release of a therapeutic such as an antiviral at a rate similar to a commercially available nondegradable implant, as well as has desirable mechanical and processing characteristics.

The period during which the device is removable might be the same or different then the period of release. In some embodiments, product can be removable for a period equal to the period of efficacy minus about six months. In preferred embodiments, the implant degrades within about six month after the period of efficacy. For example, in some embodiments, agent release occurs over a period of preferably 12-18 months, but implant degradation occurs over a period of 18 to 36 months. In other embodiments, the implant can be removable for up to a year or up to 18 months, with 18 months or 24 months of efficacy. In a particular embodiment, the implant is removable for up to a year with 18 months of efficacy. In this context, removable or removability means the implant retain sufficient mechanical properties to be removed (e.g., before or after release is complete), typically using forceps to physically withdraw the implant from under the skin. In some embodiments, degradation is complete within 6 months of the end of release.

The preferred polymer has intermediate amounts of the co-polymer (i.e., the % DO is 7-50%, such as 39-50%). The range of agent loading can be, for example, about 5% to about 30%, or higher, with approximately 25 weight % providing a preferred release. The implants are mechanically strong after incubation for nearly one year in buffered saline solutions, indicating that they will be mechanically robust for removal up to this period. In some embodiments, the implant includes a pure polymer coating and/or a pure polymer core that can be used to further modulate and tune agent release.

In some embodiments, the formulation is microparticles having co-encapsulated two or more anti-hiv agents (or a mixture of particles with one drug and others with a different drug.

As demonstrated by the examples, long term release was obtained using an implant comprised of copolymers of ω-pentadecalactone and p-dioxanone, poly(PDL-co-DO) or poly(ethylene brassylate-co-dioxanone) (poly(EB-co-DO), a class of biocompatible, biodegradable materials, as well as with PLGA particles. The study describes the pharmacokinetics and efficacy of this additive long-acting combination in humanized mice as a LA implant and nanoformulation. Both the LA implant and PLGA-based nanoformulation were able to deliver sustained therapeutic levels of agents effectively after administration and were able to suppress viremia for up to 42 days while providing protection to CD4⁺ T cells. In a second embodiment, additive antiviral activity of Compound I with temsavir, the active component of Fostemavir (RUKOBIA®), a first-in-class HIV attachment inhibitor recently approved by the FDA, in the agent combination study.

In a preferred embodiment, the composition is a combination of NRTI and NNRTI anti-HIV compounds as long-acting implants or as an extended release poly(lactide-co-glycolide) (PLGA)-based nanoformulation. Previous studies showed potent synergy with the NRTI, EFdA, and a preclinical candidate NNRTI, Compound I, which is a catechol diether:

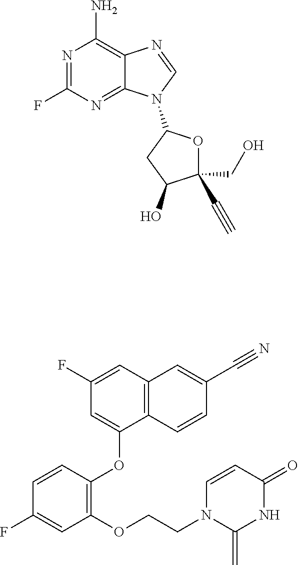

(A) 4′-Ethynyl-2-fluoro-2′-deoxyadenosine (EFdA) (B) Compound I

Development of Compound I was guided by mechanistic studies and computational design leading to enhanced pharmacological properties, agent resistance profiles, and a wide margin of safety relative to the current FDA approved NNRTIs such as efavirenz and rilpivirine. Compound I was active in the nanomolar range against wild-type HIV-1 strains and common agent resistant variants including Y181C and Y181C, K103N and demonstrated additive antiviral activity with existing HIV-1 agents and clinical candidates such as EFdA. Compound I exhibits antiviral activity that was greater than additive (synergy) with temsavir, the active component of Fostemavir (RUKOBIA®). Other polymer devices, including polymeric particles such as poly(lactide-co-glycolide) (“PLGA”) nanoformulation of Compound I enabled sustained maintenance of plasma agent concentrations and antiviral efficacy for 3-5 weeks after a single dose.

The poly(PDL-co-DO) implants were formulated independently with the EFdA and Compound I, since each has distinct physiochemical properties. Compound I is hydrophobic while EFdA is hydrophilic. For comparison, we also prepared nanoparticles composed of PLGA incorporating either EFdA or Compound I. The current report describes the evaluation of the NRTI/NNRTI combination of EFdA and Compound I as long-acting poly(PDL-co-DO) implants and PLGA-based long-acting nanoformulations in terms of pharmacokinetics and antiviral efficacy in a Hu-PBL mouse model of HIV infection. These studies with both the implants and long-acting nanoformulations of the EFdA/Compound I combinations showed sustained levels of each agent to provide protection of CD4⁺ T cells and suppression of plasma viral loads (PVLs) for almost 2 months.

In another preferred embodiment, the implant contains poly(EB-co-DO) formulated with anti-inflammatories (e.g., dexamethasone), progestins (e.g., levonorgestrel), or integrase inhibitors (e.g., dolutegravir). The poly(EB-co-DO) implants are well tolerated after subcutaneous implantation in mice, demonstrating their suitability for safe use for long-term drug release.

Also described are methods of using the implants and/or particles for delivery and/or controlled release of agents to cells, tissues, and/or organs, in drug delivery platforms. The implants or compositions thereof, can be used in combination therapy settings, to deliver two or more types of drugs that belong to the same or different therapeutic class and display the same or different mechanism of action. For example, one type of drug can be encapsulated, while a second drug is provided as free or soluble drug, or in a different carrier or dosage form. The drug-loaded implants or compositions thereof demonstrate effective antiviral efficacy e.g., reduction in HIV viral loads.

### BRIEF DESCRIPTION OF THE DRAWINGS

FIGS. 1A, 1B, and 1C show side, bottom, and top views (respectively) of an exemplary device for making a concentrated polymer agent solution (100). FIGS. 1D and 1E show side views of alternative versions of an exemplary top plunger (110a, 110b). FIG. 1F shows a side view of an exemplary bottom plunger (120).

FIGS. 2A, 2B, and 2C show side, top, and bottom views (respectively) of an exemplary heavy plunger (200a), configured to insert into a baking mold (200b). FIGS. 2D, 2E, and 2F show side, top, and bottom views (respectively) of an exemplary baking mold (200b).

FIGS. 3A-3D are cross-sectional views of exemplary implant embodiments including standard implants (FIG. 3A), coated implants having an agent-free sheet or film coating around a standard agent-containing implant (FIG. 3B), core implants having an agent-free core within a standard agent-containing implant (FIG. 3C), and coated+core implants having an agent-free core and coating (FIG. 3D).

FIGS. 4A-4E. Experimental design (FIG. 4A). Hu-PBL mice were co-implanted with Compound I and EFdA implants or co-administered with Compound I and EFdA nanoparticles. Experiment was conducted once.

Serum concentrations of Compound I (FIG. 4D) and EFdA (FIG. 4E) from in Hu-PBL mice (n=4) co-implanted with Compound I and EFdA implants. Individual measurements are shown. Suppression of systemic HIV infection in Hu-PBL mice by total plasma viral load determined by RT-PCR (FIG. 4B) in HIVJR-CSF-infected NSG mice and FACS analysis showing the percentage of human CD4+ among total CD3+ T cells of peripheral blood (FIG. 4C). Data points represent ±SD.

FIGS. 5A-5E Experimental design (FIG. 5A). Hu-PBL mice were co-administered with Compound I and EFdA nanoparticles. Experiment was conducted once. Serum concentrations of Compound I (FIG. 5B) and EFdA (FIG. 5C) from in Hu-PBL mice (n=2) co-administered with Compound I and EFdA nanoparticles. Individual measurements are shown. Suppression of systemic HIV infection in Hu-PBL mice by total plasma viral load determined by RT-PCR in HIVJR-CSF-infected NSG mice (FIGS. 5D, 5F) FACS analysis showing the percentage of human CD4+ among total CD3+ T cells of peripheral blood (FIGS. 5E, 5G). Data points represent ±SD.

FIG. 6 shows in vitro inhibition of HIV-1 infection with serum from Hu-PBL mice undergoing Compound I/EFdA combination therapy supplied through implants or NPs. The values are mean±SD from a single experiment involving triplicate measurements.

FIG. 7 is a graph showing additive inhibition of HIV-1 replication by combinations of compound I with Temsavir analyzed by isobologram analysis. The dotted lines from the upper left corners to the lower right corners indicate that the two agents are additive, and the curves below the lines indicate that agents are synergistic. The results are from three experiments involving triplicate determination.

FIGS. 8A and 8B are point graphs showing percent conversion of ethylene brassylate (EB) and dioxanone (DO) versus time for the reaction at 80° C. (calculated using 1H NMR) (FIG. 8A), and weight-average molecular weight and PDI versus time for the copolymerization reaction (determine in DCM by GPC against polystyrene standards) (FIG. 8B).

FIGS. 9A, 9B, and 9C are line graphs showing cumulative in vitro DEX release from 1 cm poly(EB-co-DO) implants of 37k, 20% DO, and 28-40% drug loading (FIG. 9A); varying drug loading from 28-40% for implants of 40k and 0% DO (FIG. 9B); and varying DO content from 0-40% for implants of 37-45k and 28% drug loading (FIG. 9C). (n=3 for Day 0-120, n=2 for Day 120+).

FIGS. 10A-10D are line graphs (FIGS. 10A and 10B) and point graphs (FIGS. 10C and 10D) effect on M_{w} with varying DO content (FIG. 10A); effect on M_{w} with drug loading at 28% and varying DO content from 0 to 40% (FIG. 10B); effect on M_{w} of DEX loading on 20% EB-co-DO polymer (FIG. 10C); and effect on M_{w} of DEX loading on EB polymer (FIG. 10D).

FIGS. 11A-11F are line graphs (FIGS. 11A, 11B, and 11D-11F) and chemical structures (FIG. 11C) showing cumulative release of implants with 28% LNG loading (FIG. 11A); cumulative release of implants with 28% DTG loading (FIG. 11B); chemical structures of DEX, LNG, and DTG with corresponding log P values (FIG. 11C); daily release of LNG implants (FIG. 11D); daily release of DTG implants (FIG. 11E); and cumulative release of DEX, DTG, and LNG implants (FIG. 11F).

FIGS. 12A-12D are column graphs showing flexural modulus of unloaded implants; ANOVA, F (4,21)=26.10, P<0.0001 (FIG. 12A); maximum flexural force before fracture of unloaded implants; ANOVA, F (4,21)=22.47, P<0.0001 (FIG. 12B); flexural modulus of 28% DEX implants; ANOVA, F (4,20)=12.95, P<0.0001 (FIG. 12C); and maximum flexural force before fracture of 28% DEX implants; ANOVA, F (4,20)=49.65, P<0.0001 (FIG. 12D).

FIG. 13 shows scanning electron microscopy (SEM) images of poly(EB-co-DO) unloaded implants with varying DO content at 5 k magnification.

FIG. 14 shows SEM images representing cross-sections of DEX-loaded poly(EB-co-DO) implants. SEM images of poly(EB-co-DO) with 28% drug loading and 0%, 7%, 20% and 40% DO over a period of four months at 5k magnification. The delineated box (last column, second and third rows) emphasizes a particular image taken at different magnification (Month 2: 40%-10k magnification, Month 4: 40%-2k magnification).

FIGS. 15A and 15B are point graphs and an array showing encapsulation thickness for average of 3 mice with implants at two to eight months (FIG. 15A) and GPC characterization of the implant at the specified time (in months) (FIG. 15B).

FIGS. 16A-16C are a line graph (FIG. 16A) and point graphs (FIGS. 16B and 16C) showing a lot of percentage loss of mass versus change in temperature using thermogravimetric analysis (FIG. 16A); melting temperature as a function of DO content in EB-co-DO (FIG. 16B); and density vs DO content of EB-co-DO polymers (FIG. 16C). In FIG. 16C, the data point at about (0, 1.16) refers to a polymer possessing a weight-average molecular weight of 52 kDA, and the remaining dots refer to polymers with weight-average molecular weights of about 40 kDa.

FIGS. 17A-17C are line graphs showing daily in vitro DEX release from 1 cm poly(EB-co-DO) implants (n=3 for Day 0-120, n=2 for Day 120+). Varying drug loading from 28%-40% for implants of 37k M_{w} and 20% DO (FIG. 17A); varying drug loading from 28%-40% for implants of 40k M_{w} and 0% DO (FIG. 17B); and varying DO content from 0%-40% for implants of 37-45k M_{w} and 28% drug loading (FIG. 18C).

FIGS. 18A-18D are column graphs showing the compressive modulus of unloaded implants; ANOVA, F (4,20)=50.89, p<0.0001 (FIG. 18A); maximum compressive force before fracture of unloaded implants; ANOVA, F (4,20)=59.00, p<0.0001 (FIG. 18B); compressive modulus of implants with varying DO contents and 28% DEX, organized by varying DO content; ANOVA, F (4,20)=18.59, p<0.0001 (FIG. 18C); maximum compressive force before fracture of implants with varying DO contents and 28% DEX, organized by varying DO content; ANOVA, F (4,20)=27.56, p<0.0001 (FIG. 18D).

FIG. 19 shows SEM images of 20% DO implants with 28%, 35%, and 40% DEX loading over a period of four months at 5k magnification. The box delineated column one, last row, emphasizes a particular image taken at 2k magnification.

FIG. 20 shows SEM images of 0% DO implants with 28%, 35%, and 40% DEX loading over a period of four months at 5k magnification.

### DETAILED DESCRIPTION OF THE INVENTION

### I. Definitions

The term “biocompatible” as used herein, generally refers to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to years.

The term “agent” as used herein, generally refers to a method or device serving to inhibit viral infection. Inhibit includes reduction of infection of cells, viral proliferation, viral release, and in the case of viruses such as HIV, viral integration into host cells.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “diagnostic agent”, as used herein, generally refers to an agent that can be administered to reveal, pinpoint, and define the localization of a pathological process.

The term “implant”, as used herein, generally refers to a device that is inserted into the body.

The term “nanoparticle”, as used herein, generally refers to a particle having a diameter, such as an average diameter, from about 10 nm up to but not including about 1 micron, preferably from 100 nm to about 1 micron. The particle can have any shape. Nanoparticles having a spherical shape are generally referred to as “nanospheres”.

Hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

The term “prophylactic agent”, as used herein, generally refers to an agent that can be administered to prevent disease.

The term “subcutaneous implantation”, as used herein, generally refers to an implantation under the skin.

The term “therapeutic agent”, as used herein, generally refers to an agent that can be administered to prevent or treat one or more symptoms of a disease or disorder. Examples include, but are not limited to, a nucleic acid, a nucleic acid analog, a small molecule, a peptidomimetic, a protein, peptide, carbohydrate or sugar, lipid, or surfactant, or a combination thereof.

### II. Compositions

The compositions described herein include implants or nano or microparticles formed of degradable polymers, having therapeutic, prophylactic and/or diagnostic agents incorporated therein or thereon, and, optionally, pharmaceutically acceptable additives. In a preferred embodiment, the implant degrades over a period of time, for example 18 months, 24 months, 30 month, 36 months, etc., thus eliminating the need for removal by a trained practitioner.

The period during which the device is removable might be the same or different then the period of release. In this context, removable or removability means the implant retain sufficient mechanical properties to be removed (e.g., before or after release is complete), typically using forceps to physically withdraw the implant from under the skin. As introduced above, some implants are never removed because they fully degrade.

In some embodiments, degradation is complete within 6 months of the end of release. For example, in some embodiments, product can be removable for a period equal to the period of efficacy minus about six months. In preferred embodiments, the implant degrades within about six month after the period of efficacy. For example, in some embodiments, agent (e.g., LNG) release occurs over a period of preferably 12-18 months, but implant degradation occurs over a period of 18 to 36 months. In other embodiments, the implant can be removable for up to a year or up to 18 months, with 18 months or 24 months of efficacy. In a particular embodiment, the implant is removable for up to a year with 18 months of efficacy.

### A. Polymers and Copolymers

i. Poly(ω-pentadecalatone-co-p-dioxanone) [poly(PDL-co-DO)]

Poly(PDL-co-DO) is a family of degradable polyester copolymers that degrade slowly in the presence of water. Poly(PDL-co-DO) copolymers are formed by ring-opening copolymerization of ω-pentadecalatone (PDL) and p-dioxanone (DO) and have the general Formula (I):

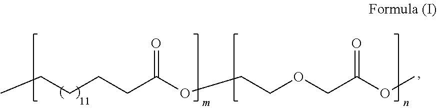

wherein n and m are independently integer values of less than or equal to about 1500.

Poly(PDL-co-DO) can be synthesized by ring-opening copolymerization of ω-pentadecalactone (PDL) with p-dioxanone (DO) as illustrated below and disclosed in Jiang et al. (Biomacromolecules 8:2262-2269; 2007):

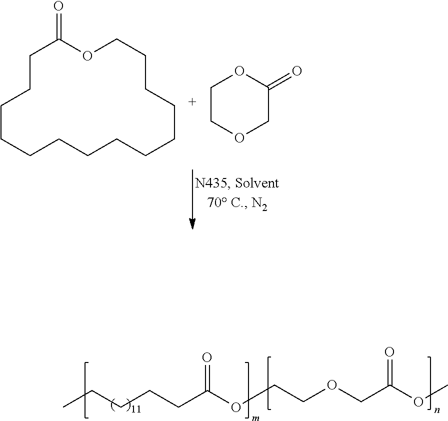

In preferred embodiments, the poly(PDL-co-DO) copolymers are enzymatically synthesized using metal-free reaction conditions.

In a non-limiting example, NOVOZYM 435 (5 wt % vs total monomer) catalyzed copolymerizations of PDL and DO co-monomers and were conducted in anhydrous toluene or diphenyl ether (200 wt % vs total monomer) at 70° C. under nitrogen for 26 hours. To synthesize copolymers with different PDL or DO contents, various PDL/DO monomer feed ratios can be used.

Poly(PDL-co-DO) copolymers have tunable biodegradability and physical properties based on the molar feed ratio of the ω-pentadecalatone and p-dioxanone comonomers used in the copolymer synthesis. In certain embodiments, the molar ratio of PDL to DO is approximately about 99:1, 95:5; 90:10, 85:15, 80:20, 75:25, 70:30, 65:35, 60:40, 55:45, 50:50, 45:55, 40:60, 35:65, 30:70, 25:75, 20:80, 15:85, 10:90, 5:95, and 1:99. In some embodiments, the molar feed ratio is in a range of about 99:1 to 1:99, or between any two values given above. In some embodiments, the poly(PDL-co-DO) copolymers have a DO mol % content of about 1, 5, 10, 15, 20, 25, 30, 35, 40, 45, 50, 55, 60, 65, 70, 75, 80, 85, 90, 95, or 99%. In some embodiments, the DO mol % content of the copolymers is in a range of about 1 to 99%, or between any two values given above. The DO mol % content can be determined from the 1H NMR of the copolymer. In embodiments, the DO mol % is between about 25% and 65% inclusive. Preferred values for this application seem to be 30-50% DO. In specific embodiments, the DO mol % content 27%, 28%, 38%, or 60%.

The copolymers described can have any molecular weight. The copolymer generally has a weight average molecular weight of at least 10,000 g/mol, at least 20,000 g/mol, at least 25,000 g/mol, at least 40,000 g/mol, at least 50,000 g/mol, at least 60,000 g/mol, at least 75,000 g/mol, at least 90,000 g/mol, at least 100,000 g/mol, at least 120,000 g/mol, at least 150,000 g/mol, at least 200,000 g/mol, at least 250,000 g/mol, at least 400,000 g/mol, at least 500,000 g/mol, or at least 750,000 g/mol. In preferred embodiments the weight average molecular weight of the Poly(PDL-co-DO) copolymers is in the range of about 25,000 g/mol to about 100,000 g/mol, more preferably about 50,000 to about 75,000 g/mol based on, for example, gel permeation chromatography (GPC) relative to polystyrene standards. The copolymers can have a polydispersity index (PDI) in the range of about 1 to about 6, more preferably about 2 to about 4, and even more preferably about 1.5 to about 2.5.

In exemplary embodiments, the copolymers have a DO content (Mol %) and Mw accordingly to the chart below.

TABLE 1
DO content (Mol %) and Mw

| DO content (Mol %) | Mw | Mw/Mn |
|---|---|---|
| 27% | 114800 | 2.2 |
| 27% | 122200 | 2.2 |
| 38% | 108000 | 2.1 |
| 38% | 108300 | 2.2 |
| 59% | 76600 | 1.8 |
| 60% | 70100 | 1.8 |

ii. Poly(ethylene brassylate-co-dioxanone) [poly(EB-co-DO)]

In some forms, the poly(EB-co-DO) has a structure:

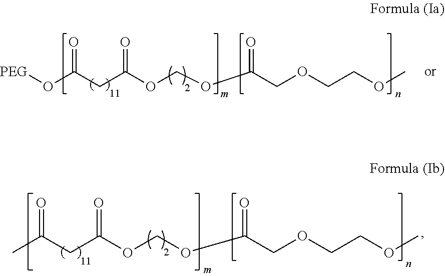

wherein, m and n are independently integers from 1 to 1500, with the proviso that m+n is at least 10 (such as from 10 to 3000). In some forms, the polymers have a weight-average molecular weight between about 10 kDa and about 150 kDa, between about 10 kDa and about 130 kDa, or between about 10 kDa and about 125 kDa, as measured using gel permeation chromatography, such as between about 15 kDa and about 25 kDa, between about 45 kDa and about 72 kDa, and between about 50 kDa and about 125 kDa.

In some forms, the mole ratio of the ethylene brassylate:dioxanone residues in the polymers is between about 95:5 and about 5:95, or between about 95:5 and about 50:50, as determined using 1H-NMR.

The copolymers described above (poly(PDL-co-DO) or poly(EB-co-DO)) can possess any degree of crystallinity. In certain embodiments, the degree of crystallinity of the copolymers is about 10, 20, 30, 40, 50, 60, 70, 80 or 90% as determined by methods such as wide-angle X-ray scattering (WAXS). The copolymers have thermal degradation temperatures of up to 425° C., which can be determined by thermal gravimetric analysis (TGA) of the copolymer. In certain embodiments, the thermal degradation temperatures of the copolymers can be up to about 300° C., 325° C., 350° C., 375° C., 400° C., or 425° C.

These polymers, methods of making, and methods of forming into implants for long term sustained release contraception, is described by W. Mark Saltzman, Elias Quijano, Fan Yang, Zhaozhong Jiang, and Derek Owen in U.S. Ser. No. 16/344,663 filed Apr. 24, 2019, entitled “Biodegradable Contraceptive Implants.

Pharmaceutically acceptable additives may be incorporated with the poly(PDL-co-DO) or poly(EB-co-DO) copolymers and compositions prepared with the copolymers prior to converting these compositions into implants. These additives can be incorporated during the formation of an agent loaded polymer composition which can be subsequently processed into implants. For example, additives may be combined with the poly(PDL-co-DO) or poly(EB-co-DO) copolymers and agent(s) and the resulting pellets or films can be compressed or extruded into implants. In another embodiment, the additives may be incorporated using a solution-based process. In a preferred embodiment, the additives are biocompatible, biodegradable, and/or bioadsorbable.

In certain embodiments, the additives may include, but are not limited to plasticizers. These additives may be added in sufficient quantity to produce the desired result. In general, these additives may be added in amounts of up to 20% by weight. Plasticizers that may be incorporated into the compositions include, but are not limited to, di-n-butyl maleate, methyl laureate, dibutyl fumarate, di(2-ethylhexyl) (dioctyl) maleate, paraffin, dodecanol, olive oil, soybean oil, polytetramethylene glycols, methyl oleate, n-propyl oleate, tetrahydrofurfuryl oleate, epoxidized linseed oil, 2-ethyl hexyl epoxytallate, glycerol triacetate, methyl linoleate, dibutyl fumarate, methyl acetyl ricinoleate, acetyl tri (n-butyl) citrate, acetyl triethyl citrate, tri (n-butyl) citrate, triethyl citrate, bis(2-hydroxyethyl) dimerate, butyl ricinoleate, glyceryl tri-(acetyl ricinoleate), methyl ricinoleate, n-butyl acetyl rincinoleate, propylene glycol ricinoleate, diethyl succinate, diisobutyl adipate, dimethyl azelate, di(n-hexyl) azelate, tri-butyl phosphate, and mixtures thereof.

iii. Polymers for Making Particles

Polymers that can be used to form the microspheres include bioerodible polymers such as poly(lactide), poly(lactide-co-glycolide), poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof, and may include small amounts of non-erodible polyurethanes, polystyrenes, polyvinyl chloride, polyvinyl fluoride, poly(vinyl imidazole), chlorosulphonated polyolefins, and polyethylene oxide. Almost any type of polymer can be used provided the appropriate solvent and non-solvent are found which have the desired melting points. In general, a polymer solution is prepared containing between 1% polymer and 30% polymer, preferably 5-10% polymer.

In the preferred embodiment, a poly(lactide) is used. As used herein, this term includes polymers of lactic acid or lactide alone, copolymers of lactic acid and glycolic acid, copolymers of lactide and glycolide, mixtures of such polymers and copolymers, the lactic acid or lactide being either in racemic or optically pure form. It is most desirable to use polylactides in the range of molecular weight up to 100,000.

The release of the antiviral agent from these polymeric systems can occur by two different mechanisms. The agent can be released by diffusion through aqueous filled channels generated in the dosage form by the dissolution of the agent or by voids created by the removal of the polymer solvent during the original microencapsulation. The second mechanism is enhanced release due to the degradation of the polymer. With time the polymer begins to erode and generates increased porosity and microstructure within the device. This creates additional pathways for agent release.

The degradation of the polymers occurs by spontaneous hydrolysis of the ester linkages on the backbone. Thus, the rate can be controlled by changing polymer properties influencing water uptake. These include the monomer ratio (lactide to glycolide), the use of L-Lactide as opposed to D/L Lactide, and the polymer molecular weight. These factors determine the hydrophilicity and crystallinity which ultimately govern the rate of water penetration. Hydrophilic excipients such as salts, carbohydrates and surfactants can also be incorporated to increase water penetration into the devices and thus accelerate the erosion of the polymer.

By altering the properties of the polymer and the properties of the dosage form, one can control the contribution of each of these release mechanisms and alter the release rate of biologically antiviral agent. Slowly eroding polymers such as poly L-lactide or high molecular weight poly(lactide-co-glycolide) with low glycolide compositions will cause the release to become diffusion controlled. Increasing the glycolide composition and decreasing the molecular weight enhances both water uptake and the hydrolysis of the polymer and adds an erosion component to the release kinetics.

The release rate can also be controlled by varying the loading of biologically antiviral agent within the microspheres. Increasing the loading will increase the network of interconnecting channels formed upon the dissolution of the agent and enhance the release of agent from the microspheres. The preferred range of biologically antiviral agent loadings is in the range of 3-30% (w/w).

Polymer hydrolysis is accelerated at acidic or basic pH's and thus the inclusion of acidic or basic excipients can be used to modulate the polymer erosion rate. The excipients can be added as particulates, can be mixed with the incorporated biologically antiviral agent or can be dissolved within the polymer.

Excipients can be also added to the biologically antiviral agent to maintain its potency depending on the duration of release. Stabilizers include carbohydrates, amino acids, fatty acids, and surfactants and are known to those skilled in the art. In addition, excipients which modify the solubility of biologically antiviral agent such as salts, complexing agents (albumin, protamine) can be used to control the release rate of the protein from the microspheres.

Stabilizers for the biologically antiviral agent are based on ratio to the protein on a weight basis. Examples include carbohydrate such as sucrose, lactose, mannitol, dextran, and heparin, proteins such as albumin and protamine, amino acids such as arginine, glycine, and threonine, surfactants such as TWEEN™ and PLURONIC™, salts such as calcium chloride and sodium phosphate, and lipids such as fatty acids, phospholipids, and bile salts.

The ratios are generally 1:10 to 4:1, carbohydrate to protein, amino acids to protein, protein stabilizer to protein, and salts to protein; 1:1000 to 1:20, surfactant to protein; and 1:20 to 4:1, lipids to protein.

Degradation enhancers are based on weight relative to the polymer weight. They can be added to the protein phase, added as a separate phase (i.e., as particulates) or can be codissolved in the polymer phase depending on the compound. In all cases the amount should be between 0.1 and thirty percent (w/w, polymer). Types of degradation enhancers include inorganic acids such as ammonium sulfate and ammonium chloride, organic acids such as citric acid, benzoic acids, heparin, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as TWEEN™ and PLURONIC™.

Pore forming agents to add microstructure to the matrices (i.e., water soluble compounds such as inorganic salts and sugars). They are added as particulates. The range should be between one and thirty percent (w/w, polymer).

### B. Antiviral Agents

In a preferred embodiment, the agent is one or more antiviral agents. Antiviral agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), sugars and polysaccharides, small molecules (typically under 1000 Daltons), lipids and lipoproteins, and biologically active portions thereof. Suitable antiviral agents have a size greater than about 1,000 Da for small peptides and polypeptides, more typically at least about 5,000 Da and often 10,000 Da or more for proteins. Nucleic acids are more typically listed in terms of base pairs or bases (collectively “bp”).

### FDA-Approved Antivirals and Regimens

Individual antiretroviral agents are described below. Dosing guides assume an absence of agent-agent interactions (except ritonavir) and normal renal and hepatic function. Dosage adjustment with renal impairment required.

### Nucleoside Reverse Transcriptase Inhibitors (NRTIs)

Abacavir (Ziagen): Dosage form: 300-mg tablet; 20-mg/mL oral solution; Adult dose: 600 mg PO qd or 300 mg PO bid

Didanosine (Videx, Videx EC) Dosage forms: 125-mg, 200-mg, 250-mg, 400-mg delayed-released capsule; 10-mg/mL powder for solution. Adult dose: >60 kg, 400 mg PO qd; <60 kg, 250 mg PO qd Lamivudine (Epivir): Dosage forms: 150-mg, 300-mg tablet; 10-mg/mL oral solution; Adult dose: 300 mg PO qd or 150 mg PO bid

Stavudine (Zerit): Dosage forms: 15-mg, 20-mg, 30-mg, 40-mg capsule; 1-mg/mL oral solution; Adult dose: >60 kg, 40 mg PO bid; <60 kg, 30 mg PO bid

Tenofovir disoproxil fumarate (DF) (Viread): Dosage forms: 150-mg, 200-mg, 250-mg, 300-mg tablets; 40-mg/g oral powder; Adult dose: 300 mg PO qd

Tenofovir alafenamide AF (various): Dosage forms: available as part of multiple coformulations; Adult dose: 25 mg PO qd; 10 mg PO qd (concomitant administration with ritonavir or cobicistat)

Zidovudine (Retrovir): Dosage forms: 300-mg tablet; 100-mg capsule; 10-mg/mL oral solution; 10-mg/mL intravenous solution; Adult dose: 300 mg PO bid or 200 mg PO tid

### Non-Nucleoside Reverse Transcriptase Inhibitors (NNRTIs)

Delavirdine (Rescriptor) Dosage forms: 100-mg, 200-mg tablets; Adult dose: 400 mg PO tid

Efavirenz (Sustiva): Dosage forms: 600-mg tablet; 50-mg, 200-mg capsule; Adult dose: 400-600 mg PO qd

Etravirine (Intelence): Dosage forms: 25-mg, 100-mg, 200-mg tablets

Adult dose: 200 mg PO bid following a meal

Nevirapine (Viramune, Viramune XR): Dosage forms: 200-mg tablet; 400-mg XR tablet; 10-mg/mL suspension; Adult dose: 200 mg PO bid (administer 200 mg qd for 2 weeks, then increase to 200 mg bid); XR, 400 mg PO qd

Rilpivirine (Edurant): Dosage forms: 25-mg tablet; Adult dose: 25 mg PO qd with a meal

Doravirine (Pifeltro): Dosage forms: 100-mg tablet; Adult dose: 100 mg PO qd

### Protease Inhibitors (PIs)

Atazanavir (Reyataz): Dosage forms: 100-mg, 150-mg, 200-mg, 300-mg capsules; 50-mg single packet oral powder; Adult dose: 400 mg PO qd or 300 mg+ritonavir 100 mg PO qd or cobicistat 150 mg PO qd

Darunavir (Prezista): Dosage forms: 75-mg, 150-mg, 300-mg, 400-mg, 600-mg tablets; Adult dose: 800 mg qd+ritonavir 100 mg PO qd or cobicistat 150 mg PO qd

Fosamprenavir (Lexiva): Dosage forms: 700-mg tablet; 50-mg/mL oral suspension; Adult dose: 700 mg bid+ritonavir 100 mg PO bid or 1400 mg PO bid or 1400 mg+ritonavir 100-200 mg PO qd

Indinavir (Crixivan): Dosage forms: 100-mg, 200-mg, 400-mg capsules; Adult dose: 800 mg PO q8h with ood

Lopinavir/ritonavir (Kaletra): Dosage forms: 100-mg/25-mg, 200-mg/50-mg tablets; 80-mg/20-mg per mL oral solution

Adult dose: 400 mg/100 mg PO bid or 800 mg/200 mg PO qd

Nelfinavir (Viracept): Dosage forms: 250-mg, 625-mg tablets, 50 mg/g oral powder; Adult dose: 1250 mg PO bid or 750 mg PO tid

Ritonavir (Norvir): Dosage forms: 100-mg tablet; 100-mg soft gelatin capsule; 80-mg/mL oral solution; Adult dose: Boosting dose for other protease inhibitors, 100-400 mg/d (refer to other protease inhibitors for specific dose); nonboosting dose (ritonavir used as sole protease inhibitor), 600 mg bid (titrate dose over 14 days, beginning with 300 mg bid on days 1-2, 400 mg bid on days 3-5, and 500 mg bid on days 6-13)

Saquinavir (Invirase): Dosage forms: 500-mg tablet; 200-mg hard gelatin capsule; Adult dose: 1000 mg+ritonavir 100 mg PO bid Tipranavir (Aptivus): Dosage forms: 250-mg soft gelatin capsule; 100-mg/mL oral solution; Adult dose: 500 mg+ritonavir 200 mg PO bid

### Integrase Inhibitors (INSTIs)

Raltegravir (Isentress, Isentress HD): Dosage forms: 400-mg tablet, 600-mg tablet; Adult dose: Isentress, 400 mg PO bid; Isentress with rifampin, 800 mg PO bid; Isentress HD, 1200 mg PO once daily

Dolutegravir (Tivicay): Dosage forms: 50-mg tablet; Adult dose: 50 mg PO once daily; with UGT1A/CY3A inducers

### Chemokine Receptor Antagonist (CCR5 Antagonist)

Maraviroc (Selzentry): Dosage forms: 150-mg, 300-mg tablets
Adult dose: 300 mg PO bid; 150 mg PO bid (CYP3A4 inhibitors±inducers); 600 mg PO bid (CYP3A4 inducers)
   Fusion inhibitor (FI)

Enfuvirtide (Fuzeon): Dosage forms: 90-mg/mL powder for injection; Adult dose: 90 mg SC bid

### Entry Inhibitor

Ibalizumab (Trogarzo) (approved only for antiretroviral treatment-experienced patients with agent resistance): Dosage forms: 150 mg/mL (200 mg/1.33 mL single-dose vial); Adult dose: First dose (single loading dose), 2000 mg IV infused over at least 30 min (begin maintenance doses 2 weeks after loading dose); maintenance doses, 800 mg IV q2Weeks infused over at least 15-30 min

### Complete Regimen Combination ARTs

ART Combination products approved as complete daily regimens, with brand name and generic names/dosages are as follows:
Cabenuva: Cabotegravir 400 mg IM+rilpivirine 600 mg IM once monthly; initiate after 1 month of lead-in therapy with cabotegravir 30 mg+rilpivirine 25 mg PO qd
Stribild: Elvitegravir (150 mg)+cobicistat (150 mg)+emtricitabine (200 mg)+tenofovir DF (300 mg) qd
Genvoya: Elvitegravir (150 mg)+cobicistat (150 mg)+emtricitabine (200 mg)+tenofovir AF (10 mg) qd
Symtuza: Darunavir (800 mg)+cobicistat (150 mg)+emtricitabine (200 mg)+tenofovir AF (10 mg) qd
Odefsey: Rilpivirine (25 mg)+emtricitabine (200 mg)+tenofovir AF (25 mg) qd; Complera: Rilpivirine (25 mg)+emtricitabine (200 mg)+tenofovir DF (300 mg) qd
Biktarvy: Bictegravir (50 mg)+emtricitabine (200 mg)+tenofovir AF (25 mg) qd
Triumeq: Dolutegravir (50 mg)+abacavir (300 mg)+lamivudine (300 mg) qd; Juluca: Dolutegravir (50 mg)+rilpivirine (25 mg) qd (Note: this is a complete once-daily regimen in adults who are virologically suppressed [HIV-1 RNA <50 copies/mL] on a stable ART regimen for ≥6 months with no history of treatment failure and no known substitutions associated with resistance); Dovato: Dolutegravir (50 mg)+lamivudine (300 mg) qd (Note: this a complete once-daily regimen for treatment-naïve adults no known substitutions associated with resistance to dolutegravir or lamivudine.)
Atripla: Efavirenz (600 mg)+emtricitabine (200 mg)+tenofovir DF (300 mg) (Note: may be use alone as a complete regimen or in combination with other ARTs.); Symfi: Efavirenz (600 mg)+lamivudine (300 mg)+tenofovir DF (300 mg) qd; Symfi Lo: Efavirenz (400 mg)+lamivudine (300 mg)+tenofovir DF (300 mg) qd
Delstrigo: Doravirine (100 mg)+lamivudine (300 mg)+tenofovir DF (300 mg) qd

Other ART combination products, with brand name and generic name/dosage, are as follows:
Descovy: Emtricitabine (200 mg)+tenofovir AF (25 mg) qd
Truvada: Emtricitabine (200 mg)+tenofovir DF (300 mg) qd
Epzicom: Abacavir (600 mg)+lamivudine (300 mg) qd
Cimduo: Lamivudine (300 mg)+tenofovir DF (300 mg) qd
Trizivir: Abacavir (300 mg)+lamivudine (150 mg)+zidovudin (300 mg) bid
Combivir: Zidovudine (300 mg)+lamivudine (150 mg) bid
Evotaz: Atazanavir (300 mg)+cobicistat (150 mg) qd
Prezcobix: Darunavir ethanolate (800 mg)+cobicistat (150 mg) qd

### Nucleoside/Nucleotide Reverse Transcriptase Inhibitors (NRTIs)

The nucleoside/nucleotide reverse transcriptase inhibitors (NRTIs) were the first agents available for the treatment of HIV infection, although less potent against HIV than non-nucleoside reverse transcriptase inhibitors (NNRTIs), protease inhibitors (PIs), and integrase strand-transfer inhibitors (INSTIs). Abacavir (ABC, Ziagen); Didanosine (ddI, Videx); Emtricitabine (FTC, Emtriva); Lamivudine (3TC, Epivir); Stavudine (d4T, Zerit); Tenofovir DF (TDF, Viread, part of the combination product Stribild and Complera); Tenofovir AF (TAF, part of the combination product Genvoya, Odefsey, and Biktarvy); Zidovudine (ZDV, and Retrovir; formerly azidothymidine [AZT])

NRTIs interrupt the HIV replication cycle via competitive inhibition of HIV reverse transcriptase and termination of the DNA chain. Reverse transcriptase is an HIV-specific DNA polymerase that allows HIV RNA to be transcribed into single-strand and ultimately double-strand proviral DNA and incorporated into the host-cell genome. Proviral DNA chain elongation is necessary before genome incorporation can occur and is accomplished by the addition of purine and pyrimidine nucleosides to the 3′ end of the growing chain. NRTIs are structurally similar to the DNA nucleoside bases and become incorporated into the proviral DNA chain, resulting in termination of proviral DNA formation. Tenofovir, lamivudine, and emtricitabine exhibit activity against hepatitis B virus (HBV) in addition to HIV and are frequently incorporated into antiretroviral regimens for patients with HIV and HBV coinfection.

Tenofovir alafenamide (AF) is a proagent of tenofovir that has high antiviral efficacy similar at a dose less than one-tenth that of the original formulation of tenofovir proagent (i.e., tenofovir disoproxil fumarate [DF]). Tenofovir AF provides lower blood levels but higher intracellular levels compared with tenofovir DF. Tenofovir AF is a substrate for p-glycoprotein and can be given at a lower dose (10 mg) when coadministered with strong p-glycoprotein inhibitors (e.g., ritonavir, cobicistat)

### Non-Nucleoside Reverse Transcriptase Inhibitors (NNRTIs)

NNRTIs exhibit potent activity against HIV-1. Efavirenz, in particular, confers the most significant inhibition of viral infectivity among the NNRTIs. The FDA approved in 2021 rilpivirine IM and cabotegravir IM as a complete once monthly regimen for treatment of HIV-1 infection in adults to replace a current stable ART regimen in those who are virologically suppressed (HIV-1 RNA <50 copies/mL) with no history of treatment failure and with no known or suspected resistance to either cabotegravir or rilpivirine. The regimen starts after 30 days of an oral lead-in therapy with rilpivirine PO and cabotegravir PO.

The highly specific NNRTI, doravirine, was approved by the FDA in 2018. Approval was based on the DRIVE-FORWARD clinical trial (n=766). Patients who were antiretroviral-naïve were randomly assigned to once-daily treatment with doravirine or darunavir 800 mg plus ritonavir 100 mg (DRV+r), each in combination with emtricitabine (FTC)/TDF or abacavir (ABC)/3TC. Treatment with doravirine led to sustained viral suppression through 48 weeks, meeting its primary endpoint of noninferiority compared with DRV+r, each in combination with FTC/TDF or ABC/3TC. At week 48, 84% of the doravirine group and 80% of the DRV+r group had plasma HIV-1 RNA of less than 50 copies/mL. All four NNRTIs exhibit activity against HIV-1 isolates. In vitro studies have shown that etravirine also has activity against HIV-2.

### Protease Inhibitors

HIV protease inhibitors (PIs) are an integral part of treatment of HIV infection. These include Atazanavir (Reyataz); Darunavir (Prezista); Fosamprenavir (Lexiva); Indinavir (Crixivan); Lopinavir/ritonavir (Kaletra); Nelfinavir (Viracept); Saquinavir (Invirase); and Tipranavir (Aptivus)

### Integrase Strand-Transfer Inhibitors

In 2017, a once daily dosage form of raltegravir (Isentress HD) was approved for adults and adolescents who weigh at least 40 kg. It is administered as a 1200 mg once-daily dose that is given as two 600-mg tablets in combination with other antiretroviral agents in patients who are either treatment-naïve or virologically suppressed on an initial regimen of raltegravir 400 mg BID. Elvitegravir/cobicistat/emtricitabine/tenofovir AF (Genvoya) was approved by the FDA to improve the renal and bone safety profile of tenofovir. Dolutegravir (Tivicay) was approved for treatment of HIV-1 infection in combination with other antiretroviral

Bictegravir is an INSTI FDA approved as a once-daily, fixed dose combination tablet with emtricitabine/tenofovir AF (Biktarvy).

The FDA approved in 2021 rilpivirine IM and cabotegravir IM as a complete once monthly regimen for treatment of HIV-1 infection in adults to replace a current stable ART regimen in those who are virologically suppressed (HIV-1 RNA <50 copies/mL) with no history of treatment failure and with no known or suspected resistance to either cabotegravir or rilpivirine. The regimen starts after 30 days of an oral lead-in therapy with rilpivirine PO and cabotegravir PO.

### Fusion Inhibitors

Fusion inhibitors (FIs) target the HIV replication cycle. Their unique mechanism of action provides additional options for therapy in patients who are highly treatment resistant.

### Chemokine Receptor Antagonists

Maraviroc (Selzentry) was approved by the FDA and was the first medication in a class of antiretroviral agents termed chemokine receptor 5 (CCR5) antagonists. It joins the fusion inhibitor, enfuvirtide, as another type of agent under the general antiretroviral treatment class of HIV-entry inhibitors. The method by which HIV binds to CD4 cells and ultimately fuses with the host cell is a complex multistep process, which begins with binding of the gp 120 HIV surface protein to the CD4 receptor. This binding induces a structural change that reveals the V3 loop of the protein. The V3 loop then binds with a chemokine coreceptor (principally either CCR5 or CXCR4), allowing gp41 to insert itself into the host cell and leading to fusion of the cell membranes. Maraviroc is a small molecule that selectively and reversibly binds the CCR5 coreceptor, blocking the V3 loop interaction and inhibiting fusion of the cellular membranes. Maraviroc is active against HIV-1 CCR5 tropic viruses.

### CD4 Post-Attachment Inhibitors

The CD4-directed post-attachment inhibitor, ibalizumab (Trogarzo), is the first medication approved for this class in March 2018. It is indicated HIV-1 infection in heavily treated adults with multiagent-resistant infection failing their current antiretroviral therapy regimen. It is used in combination with the patient's current ART regimen.

Ibalizumab is a humanized monoclonal antibody (mAb) that binds to extracellular domain 2 of the CD4 receptor. The ibalizumab binding epitope is located at the interface between domains 1 and 2, opposite from the binding site for major histocompatibility complex class II molecules and gp120 attachment. Ibalizumab does not inhibit HIV gp120 attachment to CD4; however, its postbinding conformational effects block the gp120-CD4 complex from interacting with CCR5 or CXCR4 and thus prevents viral entry and fusion.

### Gp120 Attachment Inhibitors

Fostemsavir (Rukobia), a proagent of temsavir, is a glycoprotein 120 (gp120) attachment inhibitor. It binds directly to the gp120 subunit on the surface of the virus and thereby blocks HIV from attaching to host immune system CD4⁺ T cells and other immune cells. It is indicated in combination with other antiretroviral agents for the treatment of HIV-1 infection in heavily treatment-experienced adults with multiagent-resistant HIV-1 infection failing their current antiretroviral regimen owing to resistance, intolerance, or safety considerations.

### Pharmacokinetic Enhancers (Boosting Agents)

Cobicistat (Tybost) is a CYP3A inhibitor. As a single agent, it is indicated to increase systemic exposure of atazanavir or darunavir (once-daily dosing regimen) in combination with other antiretroviral agents. It is more commonly used in co-formulations with these protease inhibitors (darunavir/cobicistat [Prezcobix], atazanavir/cobicistat [Evotaz]) or as a component of several elvitegravir-containing fixed dose combinations (elvitegravir/cobicistat/emtricitabine/tenofovir DF [Stribild], elvitegravir/cobicistat/emtricitabine/tenofovir AF [Genvoya]).

Cobicistat may be used for treatment-naïve or experienced patients (without darunavir resistance-associated substitutions). The dosage is 150 mg PO once daily when used with atazanavir (300 mg PO once daily), darunavir (800 mg PO once daily) or elvitegravir (150 mg PO once daily).

Ritonavir is also a potent CYP3A4 inhibitor that is in many combination productions and included in many HIV treatment regimens to augment systemic exposure to other antiretroviral agents.

### Combination of NRT1 and NNRTI Compounds

An NRTI and an NNRTI anti-HIV compounds were combined into long-acting implants and as an extended release poly(lactide-co-glycolide) (PLGA)-based nanoformulation. It is known that the NRTI, EFdA, and a preclinical candidate NNRTI, Compound I, which is a catechol diether, act together to produce a synergistic effect:

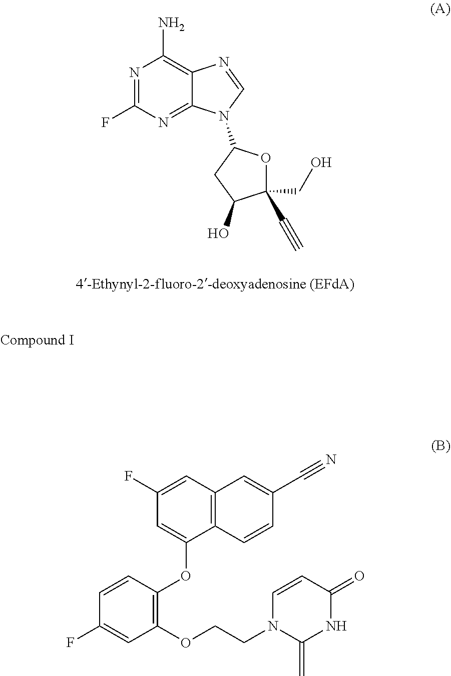

Development of Compound I was guided by mechanistic studies and computational design leading to enhanced pharmacological properties, agent resistance profiles, and a wide margin of safety relative to the current FDA approved NNRTIs such as efavirenz and rilpivirine. Compound I also demonstrated favorable pharmacokinetics and absorption, distribution, metabolism, excretion, toxicity (ADME-Tox) profile as well as antiviral efficacy in a humanized mouse model for HIV-1 (Kudalkar S N, (Proc Natl Acad Sci USA 115 (4): E802-E811 (2018); Kudalkar S N, Mol Pharmacol 91 (4): 383-391 (2017)). Compound I showed no inhibition of the HERG ion channel which might prolong the Q-T interval leading to cardiotoxicity which has limited the dosing of the NNRTI rilpivirine (Kudalkar S N, Mol Pharmacol 91 (4): 383-391 (2017)). Compound I is active in the nanomolar range against wild-type HIV-1 strains and common agent resistant variants including Y181C and Y181C, K103N and has demonstrated additive antiviral activity with existing HIV-1 agents and clinical candidates such as EFdA. Compound I exhibits antiviral activity that was greater than additive (synergy) with temsavir, the active component of Fostemavir (RUKOBIA®).

EFdA has been shown to be one of the most potent NRTIs evaluated and the clinical trials conducted by Merck have shown very good results (Markowitz M, Current opinion in HIV and AIDS 13 (4): 294-299 (2018); Stoddart C A, Antimicrobial Agents and Chemotherapy 59 (7): 4190-4198 (2015)). Previous studies have shown that EFdA is a poor substrate for the host polymerase, human mitochondrial DNA polymerase that has been associated with long-term toxicity of other NRTIs.

### Polymeric Implants of a NNRTI and NRTI

Compound I and EFdA were chosen as the NNRTI and NRTI, respectively, based upon their optimal pharmacological ADME-Tox properties, potent and additive antiviral efficacy in suppressing viral replication. For designing implants that would deliver the NRTI (EFdA) and the NNRTI (Compound 1), a polymer that would be biodegradable but maintain structural integrity during degradation and also allow for ready removal should issues with toxicity arise was selected. Accordingly, copolymers of ω-pentadecalactone and p-dioxanone, poly(PDL-co-DO), a class of biocompatible, biodegradable materials, was used to make the implants. These polymers have unique physical properties and are particularly well suited for providing long-term sustained release, not achievable with other polymers that degrade via hydrolysis such as PLGA and other polyesters (Jiang Z, Biomacromolecules 8 (7): 2262-2269 (2007)).

Poly(PDL-co-DO) implants were formulated independently with the EFdA and Compound I, since each has distinct physiochemical properties. Compound I is hydrophobic while EFdA is hydrophilic. Nanoparticles composed of PLGA incorporating either EFdA or Compound I were also prepared. The examples demonstrate that the NRTI/NNRTI combination of EFdA and Compound I as long-acting poly(PDL-co-DO) implants and PLGA-based long-acting nanoformulations show long term efficacious pharmacokinetics and antiviral efficacy in a Hu-PBL mouse model of HIV infection. These studies with both the implants and long-acting nanoformulations of the EFdA/Compound I combinations showed sustained levels of each agent to provide protection of CD4⁺ T cells and suppression of plasma viral loads (PVLs) for almost two months.

Poly(PDL-co-DO) is semicrystalline over the entire range of copolymer compositions; altering the copolymer composition allows its degradation rate, agent release rate, and other physical properties to be tuned for specific biomedical applications. Importantly, degradation is slow enough to allow production of a degradable device that remains mechanically strong for at least 12 months. Films of poly(PDL-co-DO) are well-tolerated after subcutaneous implantation and poly(PDL-co-DO) can be formulated into particles that slowly release doxorubicin or siRNA. Poly(PDL-co-DO) also can be engineered into a degradable contraceptive implant that provides consistent release of levonorgestrel (LNG) for periods of over two years.

Imaging agents such as radioopaque compounds may also be incorporated to facilitate localization at the time of placement or removal.

### III. Methods of Making Devices

### A. Methods of Making Implants

The biodegradable implants formed of a copolyester encapsulating one or more therapeutic such as an antivirals can be formed by molding, compression, extrusion, or other polymer processing methods. In some embodiments, the implant is formed using a mold. The mold can be made from any suitable material.

The implants can have a rod, bar, disc, or plate shape of any dimension which is suitable for implantation into the body. The implants formed by the methods described do not include or form nanoparticles, nor are they injectable using a needle (gauge 14 or smaller) and syringe.

In some embodiments, the implants are cylindrical rods of various lengths. In certain embodiments the length of the cylindrical rod shaped implants is in the range of about 0.1 to about 20 mm and the diameter of the implants is in the range of about 0.1 to about 5 mm. In preferred embodiments, the cylindrical rod shaped implants are about 13 mm in length and 2.4 mm in diameter.

In one non-limiting method of making the biodegradable implant, the method includes the steps of:
(1) preparing a solution of a biocompatible polyester copolymer and a therapeutic such as an antiviral in a suitable solvent to form a composition including the copolymer and the agent;
(2) loading the composition into a mold; and
(3) forming the implant.

In some embodiments step (1) includes drawing the solution of the composition into a pipette and removing the solvent, such as by evaporation at atmospheric pressure or under vacuum, to form one or more pellets comprising the copolymer and the agent.

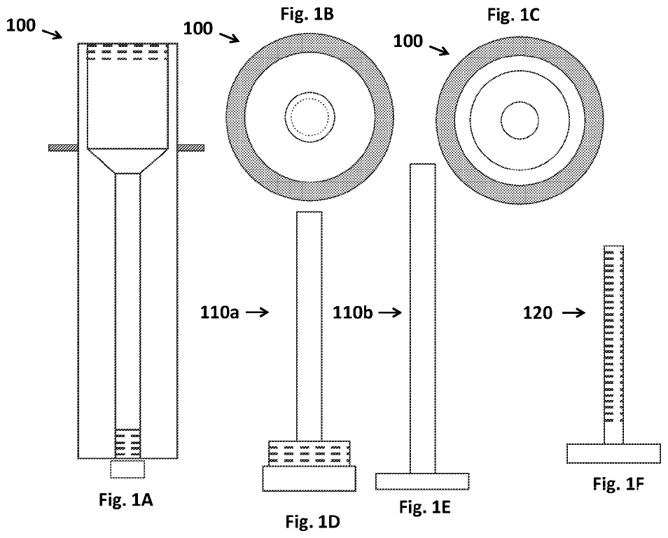

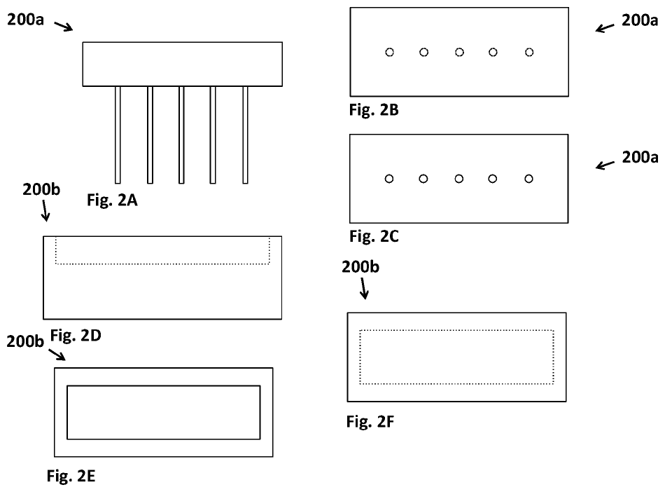

In certain embodiments the mold of step (2) is an evaporation or baking mold. Non-limiting examples of molds which can be used according to the methods described are shown in FIGS. 1A-1F and 2A-2F. Step (2) can further include spinning or centrifuging the evaporation mold, at a suitable centrifugal force, while under vacuum to remove the solvent in order to form one or more pellets comprising the copolymer and the agent. In certain embodiments, step (2) can further include a baking step performed under an inert atmosphere. Baking is typically carried out at a temperature in the range of about 50° C. to about 100° C. for a period of time in the range of about 0.1 to about 24 hours, more preferably 1 to 10 hours.

In step (3) of the general method described above, forming the implant may include the application of pressure to compress the composition, which is formed of the one or more pellets formed in either step (1) or (2), in the mold and which can be followed by a baking step typically performed under an inert atmosphere. Baking is typically carried out at a temperature in the range of about 50° C. to about 100° C. for a period of time in the range of about 0.1 to about 24 hours, more preferably 1 to 10 hours.

In certain embodiments, step (1) includes forming the composition into a film. In a non-limiting example, a film may be formed by adding the solution of step (1) into water and removing the solvent (such as by rotary evaporation) to afford a film. The resulting film can be optionally subjected to lyophilization to remove excess water.

In certain embodiments, the implant formed in step (3) is formed by extrusion of the composition. Extrusion is a particularly preferred method of manufacture, and empirical evidence shows that the materials form suitable implants when formed in this way.

Implants can be produced with or without drugs loaded with them, using the ethylene brassylate-co-dioxanone polymers described herein. In some forms, the implants have a dimension between 1 mm and 5 cm. In some forms, the ethylene brassylate-co-dioxanone polymers were used to produce unloaded and drug-loaded implants according to an established melt-molding technique (W. Saltzman, E. Quijano, F. Yang, J. Jiang, D. Owen, Biodegradable Contraceptive Implants, 2020).

In preferred embodiments of the method, the biocompatible polyester copolymer forming the implant is poly(ω-pentadecalactone-co-p-dioxanone) or (poly(EB-co-DO), as described above, and the prophylactic agent is the agent levonorgestrel (LNG).

Other non-limiting methods of making the biodegradable implants encapsulating agent include, for example:

i. Micropipette Loading of Agent/Polymer

In this method the polymer and the agent are dissolved in dichloromethane (DCM). The solution is drawn into a glass micropipette, and the DCM is evaporated to form polymer/agent pellets. The pellets can be injected into a mold, preferably a TEFLON® mold, compressed with a steel rod, and baked under argon protection. Preferably, the implant is then cooled overnight after baking.

ii. Centrifugal Loading of Agent/Polymer

The polymer and agent are dissolved in DCM. The polymer/agent solution is then loaded into a custom-built evaporation insert and centrifuged under vacuum to produce a pellet. The pellet is injected into a mold, such as a Teflon mold, for example. The material is baked under argon protection and compressed with a steel rod. Preferably, the implant is then cooled overnight after baking.

iii. Film Production and Manual Loading of Agent/Polymer

The polymer and agent are co-dissolved in DCM. The polymer/agent solution is then added to water in a rotary evaporator. The polymer/agent film precipitates out of solution as the DCM evaporates. The material can then be recovered, lyophilized and loaded into a mold. The material is baked in the mold under argon protection for two hours and then immediately compressed with a modified heavy plunger.

The foregoing is exemplary. It is understood that other methods can be used, and that modifications will be required to scale up production.

iv. Coated Implants, Core Implants, and Coated+Core Implants

The implants can include a coating or film, a core, or a combination thereof. The coating and the core can be agent-containing or agent-free. For example, implants can contain an agent-free (also referred to herein as “pure polymer”) core to shorten the final agent release tail. Additionally, or alternatively, the implants can include an agent-free (i.e., pure polymer) coating to reduce initial burst release. Exemplary, non-limiting embodiments are illustrated in FIGS. 1A, 1B, and 1C show side, bottom, and top views (respectively) of an exemplary device for making a concentrated polymer agent solution (100). FIGS. 1D and 1E show side views of alternative versions of an exemplary top plunger (110a, 110b). FIG. 1F shows a side view of an exemplary bottom plunger (120).

In an exemplary method for pure polymer core fabrication, polymer is loaded inside a baking mold (e.g., (d=1 mm)) and baked to create pure polymer core. FIGS. 2A, 2B, and 2C show side, top, and bottom views (respectively) of an exemplary heavy plunger (200a), configured to insert into a baking mold (200b). FIGS. 2D, 2E, and 2F show side, top, and bottom views (respectively) of an exemplary baking mold (200b).

Coated implants can be fabricated by preparing a polymer sheet and coating the polymer sheet on the implant.

In an exemplary method for polymer sheet fabrication, a polymer sheet is formed by dissolving PDL-co-DO in chloroform, pouring the solution into glass Petri dish, allowing chloroform to evaporate (e.g., over night at room temperature), and harvesting the PDL-co-DO sheet.

In an exemplary method for coating polymer sheets onto implants, an implant is sandwiched between two polymer sheets and placed inside a baking mold and allowed to bake (e.g., for 10 min at 70-80° C. in atmosphere pressure with argon protection). In some embodiments, the coating sheet includes agent. In an exemplary embodiment PDL-co-DO (DO context of, for example, 36%) polymer and LNG (LNG loading is kept constant at, for example, 20%).

In preferred embodiments, the coating is agent-free and is effective to reduce any initial burst effect of agent released from the implant relative to an uncoated implant. After coating, excess polymer can be cut from the implant. In an exemplary method for preparing pure polymer film, pure polymer is loaded onto aluminum foil at the base of a mold and compressed using a plunger. The polymer-containing mold is baked (e.g., at 90-100° C. for about 1 hr), removed from the heat, compressed, and allowed to cool (e.g., overnight). Similarly, agent-loaded film can be prepared by mixing polymer and agent, loading the mixture inside the mold on a sheet of aluminum foil, compressing the mixture using the plunger and baking it (e.g., for about 1 h at 90-100° C.). The film can be compressed again and cooled down.

The mold and plunger/compressor can be used in this fashion to tune the film to various desired thicknesses.

The films can be formed by solvent evaporation from solutions of polymer/solvent which can be referred to by weight/volume as a percentage. For example, in some embodiments, the coating is between about 5% and 50%, or between about 10% and 30%, or between about 15% and 25%, or about 15, 16, 17, 18, 19, 20, 21, 22, 23, 24, or 25% PDL-co-DO. Higher and lower percentages are also envisioned. In particular embodiments, the percentage is between about 5% and about 60% inclusive, or any discrete integer between about 5% and about 60% inclusive.

Generally, the desired thickness of the coating and/or core can be one that is effective to reduce release rates, and/or reduce or eliminate a burst or tail of non-linear agent release from the implant. The burst or tail can be at the beginning, end, or an intermediate stage of agent release. In some embodiments, the coated and/or cored implants maintain zero order release kinetics despite having an agent-free coating and/or core.

In some embodiments, the thickness of a coating is at least about several hundred microns. For example, in some embodiments, the thickness is from around 0.3 mm to 1 mm, or about 0.4 mm to about 0.6 mm. These thickness ranges are appropriate for 1, 2, 3, and 4 cm implants.

In some embodiments, a pure polymer sheet is fabricated by a casting method (e.g., casting the thick pure polymer solution on to glass petri dish, allow organic solvent to dry and harvest the sheet).

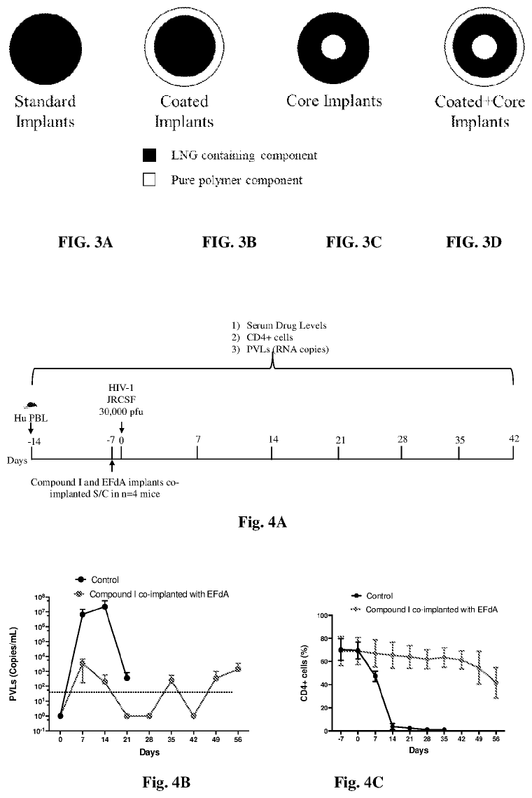

FIGS. 3A-3D are cross-sectional views of exemplary implant embodiments including standard implants (FIG. 3A), coated implants having an agent-free sheet or film coating around a standard agent-containing implant (FIG. 3B), core implants having an agent-free core within a standard agent-containing implant (FIG. 3C), and coated+core implants having an agent-free core and coating (FIG. 3D).

In some embodiments, the DO mol % content in two or more of the coating, the core, and the implant are the same or different. For example, in some embodiments, the DO mol % of the coating is lower or higher than DO mol % of the implant. The DO mol % content discussed in greater detail about with respect to the implant, can thus also be utilized in the coating and/or the core. In a particular exemplary embodiment, the DO mol % of the coating and/or the core is between about 25% and about 75%. In a particular embodiment, the DO mol % of the coating is about 37%, which may be a preferred DO mol % to prevent initial burst.

In some embodiments, the film or coating is prepared with a three-part molding system: from top to bottom are the plunger that can be used to compress the polymer into shape, the middle molding chamber that is a lumen, and the bottom platform (base). The middle molding chamber can be separate from the base platform to allow easy access and harvest of the fabricated polymer sheet, and to allow insertion of different materials (i.e. aluminum sheet, paper) as a layer for polymer sheet to form on.

Mold use is important to fabrication. Molds can be used to fabricate implants on a small scale, and can be used to fabricate implants on a larger scale, using multiple molds at once. The implant may be fabricated through, for example, twin screw, or hot-melt extrusion.

Pure polymer cores can be prepared by loading polymer inside a baking mold (e.g., (d=1 mm)) and baking to create pure polymer core.

### B. Methods of Making Micro and Nanoparticulates Formation of Microspheres.

As used herein, the term “microspheres” includes microparticles and microcapsules (having a core of a different material than the outer wall), having a diameter from nanometers range up to 5 mm. Nanoparticles are preferably between 100 and 450 nm. Microparticles are typically between 1 micron and a few hundred microns.

As characterized in the following examples, microspheres can be fabricated from different polymers using different methods.

The most common method is to use an emulsion to form particles. Polymer is dissolved in a solvent such as methylene chloride and the solution is added to a non-solvent to form particles. In one embodiment, solvent evaporation is used to precipitate the dissolved polymer into the particles; in another the solvent is removed by spray drying or evaporation or lyophilization. In solvent evaporation the polymer is dissolved in a volatile organic solvent, such as methylene chloride. The agent (either soluble or dispersed as fine particles) is added to the solution, and the mixture is suspended in an aqueous solution that contains a surface antiviral agent such as poly(vinyl alcohol). The resulting emulsion is stirred until most of the organic solvent evaporated, leaving solid microspheres. Microspheres with different sizes (1-1000 microns) and morphologies can be obtained by this method. This method is useful for relatively stable polymers like polyesters and polystyrene. Labile polymers, such as polyanhydrides, may degrade during the fabrication process due to the presence of water. For these polymers, methods which are performed in completely anhydrous organic solvents, are more useful. In hot melt encapsulation, the polymer is first melted and then mixed with the solid particles of dye or agent that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decantation with petroleum ether to give a free-flowing powder. Microspheres with sizes between one to 1000 microns are obtained with this method. The external surfaces of spheres prepared with this technique are usually smooth and dense. This procedure can be used to prepare microspheres made of polyesters and polyanhydrides. However, this method is limited to polymers with molecular weights between 1000-50,000. In solvent removal, the agent is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make microspheres from polymers with high melting points and different molecular weights. Microspheres that range between 1-300 microns can be obtained by this procedure. The external morphology of spheres produced with this technique is highly dependent on the type of polymer used.

All of the foregoing is exemplary. It is understood that other methods can be used, and that modifications will be required to scale up production.

### IV. Methods of Use

Methods of using the implants and particles for delivery and/or controlled release of agents to cells, tissues, and/or organs, in drug delivery platforms are also provided. The implants or compositions thereof, can be used in combination therapy settings, to deliver two or more types of drugs that belong to the same or different therapeutic class and display the same or different mechanism of action. For example, one type of drug can be encapsulated, while a second drug is provided as free or soluble drug, or in a different carrier or dosage form.

In preferred embodiments, the implants are effective in treating a viral infection, e.g., HIV and Acquired Immune Deficiency Syndrome (AIDS).

Implants are to be placed under standard surgical procedures, which may include laparoscopically or injection through a catheter or small incision in the skin in the same manner as the other controlled agent release implants that are nondegradable, such as NORPLANT® and JADELLE®. In preferred embodiments, implants are implanted subcutaneously or subdermally, for example, using a trocar. A local anesthetic is applied and an incision is made down to the subcutaneous layer of the skin. This creates a pocket in which the implant will be inserted. The incision is stitched shut after placement of the implant. Application of surgical tape can minimize movement of the implant while the skin fuses around the implant. In some embodiments, the implant is administered via a large gauge needle or a trocar.

Microparticles can be injected intramuscularly to maximize controlled sustained release.

The precise dosage administered to a patient will depend on many factors, including the length of time over which agent is to be released and the specific agent being released. For example, in some forms, the timing of release and amount released would be that amount, which is therapeutically effective, and may range from 3-4 weeks to a year or more. Release may occur from one or more reservoirs, as needed, and at such intervals as required to produce the effective local or systemic dosage.

The present invention will be further understood by reference to the following non-limiting examples.

### EXAMPLES

### Example 1: Implants and NPs for Sustained Release of Anti-HIV Agents

### Materials and Methods

### Materials.

EFdA was custom synthesized by WUXI, Shanghai, China. Temsavir was available for purchase from MedChemExpress, Monmouth Junction, NJ. Synthesis of Compound I has previously been reported (Bollini M, Bioorganic & medicinal chemistry letters 23 (18): 5213-5216 (2013); Lee W G, ACS Med Chem Lett 5 (11): 1259-1262 (2014)). Poly(d,1 lactic-coglycolic-acid), 50:50 with inherent viscosity 0.95-1.20 dL/g, was purchased from DURECT Corporation. ω-pentadecalactone (PDL) was purchased from Sigma and p-dioxanone (DO) was purchased from BOC sciences. Poly(PDL-co-DO) was synthesized and characterized as reported previously. (Jiang Z, Biomacromolecules 8 (7): 2262-2269 (2007))

### Fabrication of Compound I and EFdA-NPs

To formulate Compound I-loaded PLGA nanoparticles (NPs), a single emulsion-solvent evaporation method was used (Kudalkar S N, (Proc Natl Acad Sci USA 115 (4): E802-E811 (2018); Kudalkar S N, Antiviral Research 167:110-116 (2019)). Briefly, 250 mg of PLGA polymer was dissolved in 5 mL of methylene chloride (DCM) overnight (for a concentration of 50 mg/mL). Separately, 32.5 mg of the Compound I was dissolved in 0.200 mL of dimethyl sulfoxide (DMSO) overnight. To create the NPs, the polymer and Compound I solutions were mixed. One mL of this solution was added dropwise to 2 mL of 5% PVA over vortex and sonicated for three cycles of 10 seconds using a probe sonicator (set at 38% amplitude). The NPs were poured into 25 mL of 0.3% PVA at room temperature for 3 hours to harden and to evaporate the DCM. Then, the NPs were pooled, collected by centrifugation at 16,000 g for 15 minutes, and washed three times in 25 mL of water. Trehalose was added as a cryoprotectant before flash freezing and lyophilizing the NPs.

Due to the hydrophilic properties of EFdA, it was necessary to formulate EFdA-loaded PLGA nanoparticles (NPs) with a water-in-oil-in-water (W-O-W) technique that has previously been used for compounds that are more water soluble. (Woodrow K A, Nature Materials 8 (6): 526-533 (2009); Mandal S KG, Antiviral Res. 156:85-91 (2018)) One hundred mg of PLGA was dissolved in 5 mL of DCM overnight (for a concentration of 20 mg/mL). To begin the inner aqueous phase of the W-O-W emulsion, 12.04 mg of EFdA compound was dissolved in 2 mL of 1 mM HEPES buffer (pH 9.0). This inner aqueous phase was added dropwise under constant magnetic stirring to the organic phase containing 5 mL of the PLGA solution and 2 mL of 1% Pluronic F-127. Briefly, the mixture was sonicated for 10 seconds using a probe sonicator (set at 38% amplitude). To form the W-O-W emulsion, the water-in-oil (WO) phase was added to 20 mL of the outer aqueous phase (1% PVA solution) and sonicated for 10 seconds. The NPs stirred at room temperature for 3 hours to harden and to evaporate the DCM. Then, the NPs were collected by centrifugation at 16,000 g for 15 minutes and washed twice in 25 mL of water. Trehalose was added as a cryoprotectant before flash freezing and lyophilizing the NPs.

### Characterization of Compound I and EFdA NPs

For nanoparticles, agent loading (DL) was determined by dissolving a known mass of lyophilized Compound I-NP in DMSO. The samples were filtered by using an Acrodisc 25-mm syringe filter with a 0.45-μm HT Tuffryn membrane (Pall Life Sciences) followed by additional dilution in acetonitrile. The samples were analyzed using the HPLC system. The limit of detection (LOD) for Compound I was 0.1 μg/ml and 0.25 μg/ml for EFdA in our HPLC analysis.

NP size, PDI, and zeta (5) potential were measured by dynamic light scattering (DLS) by resuspending 0.05 mg NPs in 1 mL deionized water using a Zetasizer Nano ZS90 (Malvern Instruments). SEM images were obtained on a Hitachi SU7000 scanning electron microscope. To measure surface charge (zeta potential), NPs were diluted in deionized water at a concentration of 0.5 mg/mL; 750 μL of solution was loaded into a disposable capillary cell (Malvern Instruments), and the charge was measured using a Malvern Nano-ZS.

### Formulation of Compound I and EFdA Containing Long-Acting Subdermal Implants

The polymer used for these implants was poly(PDL-co-DO) with 40% p-dioxanone (DO) content (mol %) and a molecular weight of 51,178 Da. The theoretical agent loading was 40% and calculated agent loading is detailed in Table 1.

### Compound I Implant:

Poly(PDL-co-DO) (180 mg) and Compound I (120 mg) were dissolved in 10 mL of a 50:50 dichloromethane (DCM): chloroform mixture. Fifty mL doubly distilled water (ddH₂O) was used to rinse a round bottom flask, leaving a small volume of ddH₂O in the flask to prevent polymer or agent from sticking to the surface of the flask. Polymer and agent solution was added to the flask and a rotary evaporator was used to completely evaporate the DCM and chloroform over ˜20 minutes. The resulting agent/polymer film was lyophilized to remove excess water. The film (˜120 mg) was then loaded into a Teflon mold and baked for 1 hr. at 90° C. under argon protection and atmospheric pressure to form implants. Immediately after baking, the implants were compressed overnight using a stainless-steel plunger. The resulting implants were 2 cm in length and approximately 100 mg in weight. The implants were then cut to 1 cm in length and weighed (Table 1).

### Efda Implants:

Poly(PDL-co-DO) (180 mg) and EFdA (120 mg) were dissolved in 10 mL of in a glass vial. A rotary evaporator with a glass vial adapter was used to completely evaporate the DCM. The resulting agent/polymer mixture was weighed into two portions, each around 120 mg. Each portion was then loaded into a Teflon mold and baked for 1 hr. at 90° C. under argon protection and atmospheric pressure to form implants. Immediately after baking, the implants were compressed overnight using a stainless-steel plunger. The resulting implants were 2 cm in length and approximately 100 mg in weight. The implants were then cut to 1 cm in length and weighed (Table 1).

### Characterization of Agent-Loaded Implants

For the EFdA implants, the sample was dissolved in 1 mL of chloroform, and 1 mL water was added to it. The mixture was vortexed and let it sit to extract the agent into the water phase. Centrifugation was carried out to separate layer of chloroform and water. The upper water layer containing the sample was removed and lyophilized to dry out the water. The dried pellet was dissolved in acetonitrile (ACN) for HPLC analysis.

For the Compound I implant, the sample was dissolved in 1 mL of DCM, and DCM was evaporated under a steady stream of nitrogen. The left-over residue was brought up in 1 mL of ACN for HPLC analysis. The limit of detection (LOD) for Compound I was 0.1 μg/ml and 0.25 μg/ml for EFdA in our HPLC analysis.

Reverse-phase HPLC was used to measure DL (%), which is defined as the measured mass of Compound I per mass of PLGA NP/implants, and EE (%), which is defined as the ratio of the compounds loaded to the total agents used for fabricating the NPs/implants:

$$DL ( \% ) = ( mass of Compound I in formulation / total mass of formulation ) \times 100$$

$$EE ( \% ) = ( mass of agent in formulation / mass of polymer used in formulation ) \times 100$$

EFdA and Compound I Implants were evaluated under an ultra-high-resolution Hitachi scanning electron microscopy (SU7000). Implants were flash frozen in liquid nitrogen. Implants were then broken in half using tweezers and cross sectioned using a razor blade to about 1 mm thickness. Samples were placed on a stub using carbon tape, with razor blade edge facing down. Samples were coated with platinum to a thickness of 5 nm using a high resolution sputter coater (Cressington, 208 HR) with rotary planetary tilt stage and thickness controller MTM-20.

### Generation of Hu-PBL Mice

NOD.Cg-Prkdcscid Il2rgtm1 Wjl/SzJ (NSG) mice were purchased from the Jackson Laboratory (Bar Harbor, ME) and bred and maintained at Yale University (New Haven, CT) according to guidelines established by the Institutional Animal Committee. All experiments were performed according to protocols approved by the Institutional Review Board and the Institutional Animal Care and Use Committee of Yale University. NSG-Hu-PBL mice were engrafted. Briefly, 10×10⁶ peripheral blood mononuclear cells (PBMC), purified by Ficoll density gradient centrifugation of healthy donor blood (obtained from the New York Blood Bank) were injected intraperitoneally in a 200 μl volume into 6- to 12-week-old NSG mice (Jackson Laboratory), using a 1 cm³ syringe and 25-gauge needle. Mice were retro-orbitally bled to collect 70-100 μl of blood. PBMC were separated by ficoll density gradient centrifugation and stained with fluor-conjugated anti-human CD45, CD3, CD4 and CD8 antibodies to confirm the cell engraftment 10 days post injection.

### Administration of Nanoparticles

Two NSG mice were injected intraperitoneally (i.p.) with Compound I-NP (190 mg/kg) and EFdA (10 mg/kg) suspended in sterile saline solution. The blood samples were collected at predetermined time points from the ocular venous plexus by retro-orbital venipuncture and used for subsequent HPLC analysis.

### Implantation Procedure

Mice were anesthetized by intraperitoneal injection of ketamine (80 mg/kg) and xylazine (12 mg/kg) in PBS (10 ml/kg) based on individual mouse body weight. After anesthetizing, approximately 1-2 cm square of the dorsal skin was shaved using an electric hair clipper, the area was cleaned with ethanol and disinfected with betadine. Using a sterile disposable surgical blade, an incision of 4-5 mm was made through the skin. Gently, 2 cm×3 cm subcutaneous pockets were created with forceps and implants loaded with EFdA and Compound I were co-inserted at the same site. The opening was sutured using absorbable sutures followed by subcutaneous administration of 0.05 mg/kg buprenorphine. The animals were kept warm using the temperature-controlled heating pads until they regained consciousness. Toxicity was evaluated by clinical observations, eageside observations (twice daily), and body weight (at least weekly).

### Pharmacokinetic Studies for Implants and NPs

To determine dose-dependent serum agent concentrations, in vivo pharmacokinetics of Compound I and EFdA implants, and Compound I-NP and EFdA-NP were investigated in Hu-PBL mice. The blood samples were collected at predetermined time points from the ocular venous plexus by retro orbital venipuncture and serum was used for subsequent HPLC analysis.

### HIV-1 Challenge Experiments in Hu-PBL Mice

Humanized mice that were surgically inserted with EFdA and Compound I loaded implants or intraperitoneally injected nanoparticles were intraperitoneally challenged with 30,000 FFU of HIV-1_{JRCSF}. The kinetics of virus infection were monitored by weekly measurements of plasma viral loads and peripheral blood CD4 T cell in longitudinal bleeding mice retro-orbitally at indicated time points. Briefly, for viral load quantification, viral RNA was isolated from plasma using the QIAamp viral RNA mini kit (QIAGEN) following the manufacturer's protocol. Followed by quantification using one-step real-time reverse transcriptase PCR assay using the following primers 5′ TGCTATGTCAGTTCCCCTTGGTTCTCT 3′ (SEQ ID NO: 1) and 5′ AGTTGGAGGACATCAAGCAGCCATGCAAAT 3′ (SEQ ID NO: 2. To analyze the CD4 T cell loss by HIV infection, PBMCs were isolated from the peripheral blood. PBMCs were stained with fluor-conjugated anti-human CD45, CD3, CD4 and CD8 antibodies and analyzed by flow cytometry to assess CD4 T cell levels.

Analysis of Antiretroviral Activity in Serum from Agent Administered Mice

Twenty-four hours prior to infection, 15,000 TZM-bl cells were seeded per well in a 96-well plate. Next day, serum collected from the EFdA- and Compound I-loaded implant and nanoparticle administered mice were diluted at 1:20 or 1:100 in complete DMEM were added to the cells. Two hours post addition of diluted mouse serum, cells were infected with HIV-1 JRCSF (multiplicity of infection, 0.1). After 48 hours, cells were lysed and luciferase activity was measured using a luciferase assay kit following manufacturer's protocol (Promega). Inhibition percentages in the agent administered (implants and nanoparticles) were calculated relative to the read outs from the control uninfected Hu-PBL mouse serum.

### Example 2: Combination Study of Compound I and Temsavir in Implants and NPs

### Materials and Methods

The combination inhibitory effects of compound I and Temsavir were tested in a two-agent combination (Ray AS, Antimicrob Agents Chemother 49 (5): 1994-2001 (2005); Kudalkar S N, Proc Natl Acad Sci USA 115 (4): E802-E811 (2018), Feng J Y, Retrovirology 6:44 (2009)). The combination inhibitory data were analyzed using isobologram analysis. For isobologram analysis, isobolograms were constructed by measuring the fractional changes in EC₅₀ (FEC₅₀s) of two anti-HIV agents when used in combination in the MT-2assay. The values on the additivity line connecting FEC₅₀ values of 1 represent an additive interaction between the two compounds, while points markedly above or below the line represent antagonism or synergy, respectively.

### Statistical Analysis

The data were plotted as a concentration-time curve using PRISM 9.0 (GraphPad Software Inc, LaJolla, CA). EC₉₀ was calculated using Graph pad prism where EC₅₀ of each compound was used along with Hill slope of 1. The predicted area under the curve (AUC) of the concentration of agent in blood serum against time (AUC predicted) was calculated based on the linear trapezoid method. AUC was also calculated for viral suppression experiment looking at plasma viral load (PVLs) and CD4+ T-cells. Wilcoxon signed rank test was performed to test any statistical significance between controls and agent treated mice.

### Results

### Development of Two-Agent Long-Acting Implants and Nanoformulations.

Compound I and EFdA were used in the preparation of four poly(PDL-co-DO) implants and PLGA loaded nanoparticles using techniques as described in methods section. All the implants were 1 cm long and white (Compound I) to yellowish white (EFdA) in color (FIG. 1C). Table 1 represents the implant weight, percent agent loading and calculated dose for each implant. The average calculated percent loading for each implant was 16.4% for Compound I and 30% for EFdA.

TABLE 2
Implant weight, calculated percent agent loading
and calculated dose for each implant

| | Implant | Agent | Dose (mg) for | Dose |
|---|---|---|---|---|
| Implants | weight (mg) | loading (%) | 20 g mouse | (mg/Kg) |
| Compound | 63.6 | 16.4 | 10.43 | 522 |
| I-1 | | | | |
| Compound | 49.4 | 16.4 | 8.10 | 405 |
| I-2 | | | | |
| Compound | 52.5 | 16.4 | 8.61 | 431 |
| I-3 | | | | |
| Compound | 32.4 | 16.4 | 5.31 | 266 |
| I-4 | | | | |
| EFdA-1 | 46.5 | 30 | 13.95 | 698 |
| EFdA-2 | 45.8 | 30 | 13.74 | 687 |
| EFdA-3 | 44.1 | 30 | 13.23 | 662 |
| EFdA-4 | 65.5 | 30 | 19.65 | 983 |

The implants were characterized using SEM to assess their morphology and uniformity. Compared to the unloaded polymer implants, which reveal a continuous grey region by SEM, the Compound I and EFdA loaded implants have regions that appeared brighter, presumably due to the presence of agent or agent crystals.

The physical characteristics of nanoparticles for Compound I-NP and EFdA-NP are listed in Table 3. The nanoformulation exhibited an average diameter of 255±4.5 nm for Compound I-NP and 255±3.7 nm for EFdA-NP, with a polydispersity index of <0.1 and an average negative zeta potential of −33.8 and −31.4 for Compound I-NP and EFdA-NP, respectively. Representative SEM images illustrated that Compound I-NP and EFdA-NP exhibited spherical shapes. For Compound I-NP an initial agent loading of 10 wt % was used resulting in agent loading of 9.7±0.8 wt % whereas for EFdA an initial agent loading of 10 wt % which resulted in 0.8±0.2 wt % (Table 3).

TABLE 3
Characterization of Compound I-NP and EFdA-NP

| | Particle | Polydispersity | Zeta | Agent |
|---|---|---|---|---|
| Nano- | size | Index | Potential | loading |
| particles | (nm) | (PDI) | (mV) | (DL %) |
| Compound | 255.4 ± 4.47 | 0.085 ± 0.018 | −33.8 ± 1.43 | 9.7 ± 0.8% |
| I-NP | | | | |
| EFdA-NP | 255.1 ± 3.67 | 0.076 ± 0.018 | −31.4 ± 2.30 | 0.8 ± 0.2% |

### Pharmacokinetics and Efficacy Studies of Compound I and EFdA Implants and Nanoparticles in Hu-PBL Mice

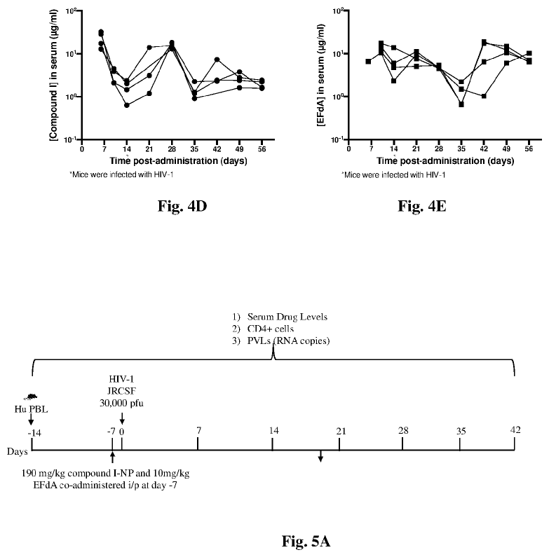

Recent studies with PLGA nanoparticles of Compound I demonstrated sustained levels of agent in serum of Hu-PBL mice for more than 30 days after a single dose. Accordingly, in vivo agent release from implants and nanoparticles were monitored in Hu-PBL mice, generated by reconstituting NSG mice with healthy human peripheral blood mononuclear cells. Given the intended use of Compound I as combination therapy with EFdA, all long-acting nanoparticles and implants were formulated independently, containing either EFdA or Compound I, but they were co-administered i.p. for nanoparticles or co-implanted subdermally for implants in Hu-PBL mice. Experiments with both formulations were performed in 4 NSG-Hu-PBL mice for implants and 2 NSG-Hu-PBL mice for nanoparticles. The mice were infected with HIV-1JR-CSF (30,000 pfu) as shown in the schemes (FIG. 4A, 5A).

No adverse events related to treatment with the implant were noted during the course of the study. Overall, there were no significant abnormalities, and the majority of observations noted were considered to be incidental, procedure related, or common findings for mice undergoing a surgical procedure. The incision sites healed normally within a few days after surgery. There was no evidence of inflammation, toxicity, or poor tolerability at the implantation sites throughout the duration of the study.

Pharmacokinetics of long-acting Compound I and EFdA implants. Implants loaded with EFdA and Compound I (Table 1) were used for in vivo analysis with the intent of achieving in vivo daily serum agent concentrations at or well above the effective concentrations (EC₅₀) for the two agents, which were 1.9 nM for EFdA and 2.8 nM for Compound I based on cellular assays.

The pharmacokinetics for the two-agent combination implants are shown in FIGS. 4D and 4E. Overall concentrations of each agent were well above the EC₅₀ values necessary to maintain viral suppression. A transient burst was noticed for all four mice containing Compound I implants at day 6 post implantation with a serum concentration in the range of 13 to 33 μg/ml (FIG. 4D). For EFdA implants in these mice, a transient burst was observed at day 10 post-implantation where serum concentration ranged from 11 to 18 μg/ml (FIG. 4E). For the majority of the measurements over the 56 d period, agent serum levels remained relatively steady. On day 14, levels were 1400 (Compound I) to 11,000 (EFdA)-fold higher than the cellular EC₅₀ values. On day 14 the mice were infected with HIV-1 JRCSF strain after which there was a transient increase in serum agent concentrations of both agents at day 21 post-implantation or 7 days post-infection. The concentration of Compound I was further increased to 16 μg/ml at day 28 or 14 days post-infection whereas EFdA concentration was maintained at same levels as day 7 post-infection. At day 35 post-implantation there was a 5 to 16-fold decrease in serum concentration of EFdA and Compound I, respectively compared to day 28. This decrease was recovered a week later and the serum levels of both agents were maintained at their pre-infection levels until day 56 post-implantation. The EFdA and Compound I implants sustained serum levels of Compound I and EFdA at greater than IC₉₀ (25.2 nM and 17.1 nM, respectively) starting at days 7-10 days after administration and maintained levels till at least 56 days which time, the experiment was terminated in accordance with the experimental timeline admissible with Hu-PBL mice. The calculated AUC₀₋ₗₐₛₜ for Compound I was 10,058+/−2074 μg*h/ml with 95% confidence interval of 5993 to 14124 μg*h/mL and for EFdA 9129+/−1323 μg*h/ml with 95% confidence interval of 6535 to 11723 μg*h/ml.

Inhibition of HIV-replication in vivo in mice receiving implants. To establish the in vivo inhibitory effects of Compound I/EFdA combination therapy, Hu-PBL mice were divided into 2 groups-control, and implant (receiving two subdermal implants, individually formulated with Compound I or EFdA). In this study, implants were surgically inserted at 14 days (D −14), prior to infection (DO) with 30,000 infectious units of HIV-1 JRCSF. Both agents were confirmed to achieve plasma concentrations of >EC₅₀ prior to being challenged with HIV-1 (FIGS. 3B and 3C). Antiviral efficacy was assessed by measuring the plasma viral loads and the levels of CD4+ T-cells post infection on weekly basis by collecting blood samples retro-orbitally. The loss of peripheral blood human CD4+ T-cells caused by the HIV-1 infection provides an indicator of pathogenic effects of viral infection. Thus, levels of human CD4⁺ cells as a percentage of total human CD45⁺CD3⁺ T cells were determined from FACS analyses in blood samples obtained on a weekly basis as described in the methods section.

HIV-RNA was readily detected in plasma of all exposed mice (FIG. 4B) at the first sampling 1 week after exposure with the levels in the control group being high (median 3.24×10⁶ copies/ml, range 0.73-19.2×10⁶ copies/ml). In contrast, the plasma viral loads (PVLs) in the implant group were 3 log units lower compared to the control group (implant group median 3.31×10³ copies/ml, range 0-7.18×10³ copies/ml). This was indicative of productive but rapidly controlled infection or residual viral RNA from the high viral inoculum used for challenge in animals in the test cohorts. Control mice maintained high concentrations of plasma HIV-1 RNA for up to 2 weeks after which, PVLs declined, concomitant with the rapid loss of human CD4+ T cells required for viral replication and titers (FIG. 4C). In contrast, viral loads fell to below the level of quantitation (LOQ, 150 copies of HIV-1 RNA/ml plasma) in 3 out of 4 mice of implant group (FIG. 4B). In the implant group, all mice continued to maintain PVLs below LOQ until 4 weeks post infection. At day 35, 2 of the 4 mice in the implant group showed the transient presence of PVLs above the LOQ, however, this level fell below LOQ at day 42-post infection. The amount of detectable PVLs in 2 of 4 mice was still 3 log units lower at day 35 as compared to the control group at the beginning of the study. Hence, 2 of the 4 mice in the implant group maintained PVLs below LOQ throughout the study duration (i.e., 8 weeks) (FIG. 4B). Plasma viral load AUC was smaller in the nanoformulation group compared to the placebo group, p=0.05 (Wilcoxon signed rank test).

The observations with the viral loads were supported by the virus-induced effects on peripheral CD4+ T cells in the different mouse groups (FIG. 4C). To begin with, the average levels of CD4⁺ T-cells on DO prior to HIV infection in both groups were approximately 70±13%. Upon infection, control mice, which did not receive any treatment, started to show a significant decline in CD4 levels and by day 14, >97% of CD4+ T cells were lost in all mice in this group. On the other hand, for mice in the implant group, >85% of CD4+ T cells were protected throughout the study relative to day 0. At the last time point, for implant (D56 pi), the only mouse with PVLs showed a significant decline in CD4+ T cells (˜32%).

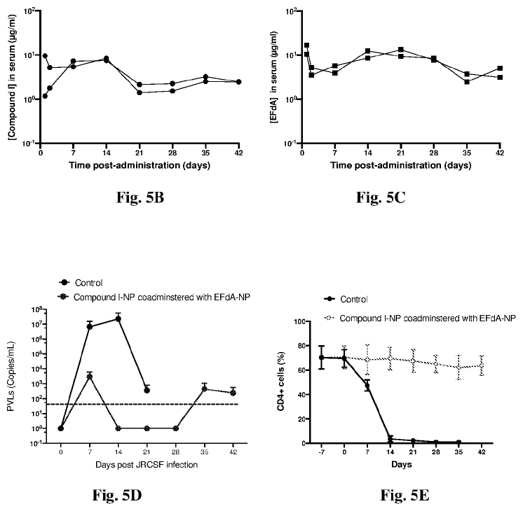

Pharmacokinetics of long-acting Compound I and EFdA nanoformulation. Nanoparticles (NPs) were administered at a dose of 190 mg/kg for Compound I and 10 mg/kg for EFdA based on the recent studies that yielded sustained in vivo agent concentrations above EC₅₀. As before, the nanoparticles also sustained serum levels of Compound I and EFdA above EC₅₀ for 42 days with a single dose administered at the beginning of the study. A transient burst with serum concentrations of 10.6 μg/ml and 13.6 μg/ml was observed 24 hours post co-administration of Compound I-NP and EFdA-NP, respectively (FIGS. 5B and 5C). There was a 50% decrease in the serum concentrations of both the agents at day 2, which was maintained until day 7. The mice were infected with HIV-JRCSF strain at day 2 and blood was collected every week to monitor the PK and efficacy of Compound I and EFdA. A 15% increase in Compound I and 21% increase in EFdA serum concentrations was observed at day 14-post infection. This level was maintained until day 21 for EFdA followed by a two-fold decline over the next three weeks. In contrast, Compound I serum concentration decreased by 7-fold at day 21 and was maintained at that level until the end of the study. The observed AUC₀₋ₗₐₛₜ for Compound I-NP was 3874+/−243.1 μg*h/ml with a clearance of 0.8 ml/min/kg. The AUC₀₋ₗₐₛₜ was 2 fold lower than the previous study whereas CL was 2 fold higher. The lower AUC₀₋ₗₐₛₜ may be due to co-administration for EFdA and the higher clearance can be attributed to the fact that the clearance is dependent on dose and calculated AUC₀₋ₗₐₛₜ. For EFdA, the observed AUC₀₋ₗₐₛₜ was 6978+/−518 μg*h/ml with clearance of 0.02 ml/min/kg.

Inhibition of HIV-replication in vivo in mice receiving nanoparticles. In this study 2 mice received a single IP dose of a mixture of nanoparticles formulated individually with Compound I and EFdA) (FIG. 5A). The agent encapsulated NPs were injected 7 days (D −7) prior to HIV-1 challenge (DO). Both agents were confirmed to achieve plasma concentrations of >EC₅₀ prior to being challenged with HIV-1. (FIGS. 5B and 5C). Similar to the implant study the antiviral efficacy was assessed by measuring the plasma viral loads and the levels of CD4+ T-cells post infection on weekly basis by collecting blood samples retro-orbitally.

The plasma viral loads (PVLs) in mice were 3 log units lower compared to the control group (median 0.72× 10³ copies/ml, range 0.71-5.19×10³ copies/ml) (FIG. 5D). This indicated that there might be productive but rapidly-controlled infection or residual viral RNA from the high viral inoculum used to challenge the mice. Control mice maintained after which, PVLs declined, concomitant with the rapid loss of human CD4+ T cells required for viral replication and titers (FIGS. 5D and 5E FIGS. 5D and 5E). In contrast to the control group, where high concentrations of plasma HIV-1 RNA were maintained for up to 2 weeks, viral loads fell to below the level of quantitation (LOQ, 150 copies of HIV-1 RNA/ml plasma) in both mice receiving the nanoformulation (FIG. 5D). PVLs in both mice were below LOQ for 4 weeks post infection. Detectable PVLs were observed in one of the 2 mice at day 35 which was maintained until the end of the study i.e., day 42 however, the levels were still 4 log units lower than what was observed in the control mice at the beginning of the study (FIG. 5D). On an average, reduction in 4 log₁₀ was seen compared to the highest PVLs attained in the control group at D14 indicating continued HIV-1 inhibition in these mice. In both mice, >85% of CD4+ T cells were protected throughout the study relative to DO (FIG. 5E). At the last time point, NP (D42 pi), the only mouse with PVLs showed a significant decline in CD4+ T cells (32% and 43% respectively). Plasma viral load AUC was smaller in the nanoformulation group compared to the placebo group, p=0.05 (Wilcoxon signed rank test).

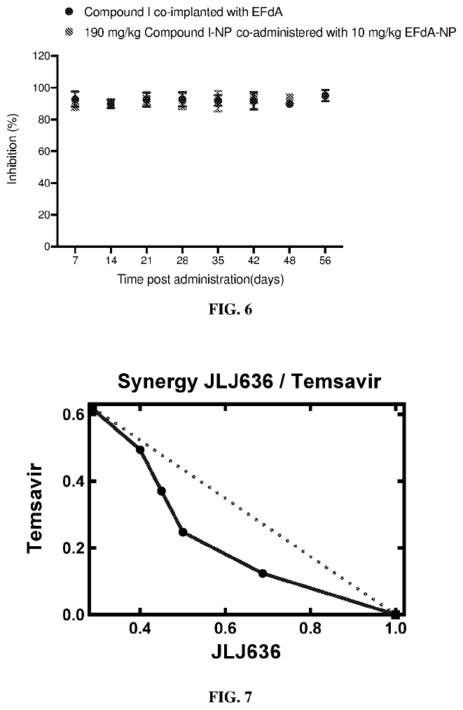

Inhibition of HIV-1 replication ex vivo. Having demonstrated sustained concentrations of Compound I and EFdA in serum of humanized mice, antiviral activity was evaluated. Serum obtained from the implanted mice demonstrated strong antiviral activity (FIG. 6). Specifically, a one hundredth-fold dilution of serum was able to block in vitro infection of TZM-bl cells with HIV-1 JRCSF by ˜90% when collected throughout the experimental timeline (at least 56 days post implant) (median 91.75%, range 89.75%-95.0%, n=4; CD4+ cells). Importantly, the activity was comparable to a 1:20 fold dilution of the serum. A 100-fold dilution of serum from long-acting NP treated mice was also able to block HIV infection by ˜90% throughout the time period of testing (median 91.50%, range 89.00%-93.0%, CD4+ cells).

### DISCUSSION

A study in humanized mice with this implant showed sustained agent levels over almost two months and a one to two log drop in viral load, however agent resistant HIV variants emerged after 19 days. In addition, several bioerodible and nonerodible implants containing EFdA as a single agent showed extended agent levels for over 6 months in rats and non human primates although no efficacy studies were reported (10). In the current study, we describe the pharmacokinetics and antiviral efficacy of a long-acting, additive two-agent combination formulated as a removable, biodegradable implant and as a single dose PLGA-based nanoparticle formulation in Hu-PBL mice. The two-agent NRTI/NNRTI combination contained EFdA as the nucleoside, selected due to its exceptional potency and lack of toxicity including mitochondrial toxicity observed with other NRTIs and Phase III clinical trials. The NNRTI in the combination was Compound I, a computationally designed preclinical candidate chosen based upon the excellent antiviral efficacy as a long-acting nanoparticle formulation, ADME-Tox and agent resistance profiles as well as additive behavior with EFdA as a two-agent combination in cell culture. The poly(PDL-co-DO) implant utilized for these studies has several desirable features for long-acting, sustained agent delivery. These attributes include: (1) biocompatible and biodegradable while maintaining structural integrity in the event removal is required due to toxicity and (2) extended long-term agent release potentially lasting for several years.

The pharmacokinetics of the two-agent combination showed sustained plasma concentrations of Compound I and EFdA from the implants in Hu-PBL mice at levels at ˜2 μg/ml (4.5 M) and 7.5 μg/ml (25 μM) approximately 178 and 1600-fold above the EC₉₀ in HIV cell culture (25.2 nM and 17.1 nM, respectively), observed at the end of the 56 day duration period of the experiment. Similar results were noted for the Compound 1/EFdA nanoparticle formulation in which the plasma levels of each agent were maintained at or above concentrations of Compound I 2.0 μg/ml (4 μM) and 4 μg/ml (13 μM), respectively at day 42 at the end of the experiment.

Potent antiviral efficacy of the two-agent Compound I/EFdA combination, delivered as individual implants in Hu-PBL mice, was observed as evidenced by the drop in plasma viral load below LOQ in 3 of the 4 mice at the of experiment 42 days post-infection and a 4 log unit drop in the remaining mouse. Since the agent concentrations in the plasma from all the mice were sufficient to completely inhibit HIV infection ex vivo, it is possible that the inadequate agent distribution to tissues in the mice caused incomplete viral suppression. The antiviral efficacy was also supported by protection of viral-induced effects on peripheral CD4+ T cells in which >85% of the population in the two-agent treatment group was preserved relative to the mice in the control group. Likewise, antiviral potency in the two mice receiving the two-agent combination as a nanoformulation was noted with viral loads below LOQ at the end of the experiment.

The current study using the two-agent combination in poly(PDL-co-DO) implants or PLGA nanoformulations demonstrates the value of two additive antiviral compounds to completely suppress viral loads and protect CD4+ T cells. This approach significantly extends previous findings with a long-acting implant containing dolutegravir as monotherapy where the onset of agent resistance was found as early as day 19 post-infection.

### Example 3: Consideration of Other Two-Agent Combinations

In the experiments described above the effectiveness of Compound I as a two-agent combination with EFdA.

With the recent FDA approval of the temsavir, the first-in-class HIV attachment inhibitor, a two-agent combination of Compound I and temsavir was formulated and tested in HIV cell culture. As illustrated FIG. 7, strong additive inhibition was observed for this two-agent NNRTI/entry inhibitor combination, which has not been previously reported.

### Example 4: Poly(Ethylene Brassylate-Co-Dioxanone) Polymers and Fabrication of Biodegradable Implants

### Materials and Methods

All chemicals were purchased from Fisher Scientific and used as received unless stated otherwise. Ethylene brassylate (EB) was purchased from Sigma Aldrich. Benzyl alcohol was distilled from calcium hydride under high vacuum. 7-Methyl-1,5,7-triazabicyclo[4.4.0]dec-5-ene (MTBD), 1,8-Diazabicyclo(5.4.0) undec-7-ene (DBU) was purchased from Tokyo Chemical Industry Co. LTD. and 2-tert-butylimino-2-diethylamino-1,3-dimethylperhydro-1,3,2-diazaphosphorine (BEMP) from Acros Organics. Benzene-de and chloroform-d were purchased from Cambridge Isotope Laboratories and distilled from calcium hydride. Experiments were conducted using pre-dried glassware in an MBRAUN or INERT stainless-steel glovebox under N₂ atmosphere. NMR experiments were conducted on a Bruker Avance III 300 MHz or 400 MHz spectrometer. Gel Permeation Chromatography (GPC) was performed at 40° C. using HPLC grade dichloromethane eluent on an Agilent Infinity GPC system equipped with three Agilent PLGel columns 7.5 mm×300 mm (5 μm, pore sizes: 10³, 10⁴, 50 Å). Mₙ and M_{w}/Mₙ were determined versus polystyrene standards (500 g/mol-3150 kg/mol, Polymer Laboratories). Differential Scanning calorimetry (DSC) experiments were conducted using a Shimadzu DSC-60A instrument, calibrated with an indium standard using aluminum pans under inert conditions.

### (i) Poly(EB-co-DO) Copolymerization Reactions

Small-scale solvent-free copolymerization using Novozyme 435. A 1 mL vial was charged with EB (270.4 mg, 1.0 mmol), DO (102.1 mg, 1.0 mmol), and a stir bar. The contents of the vial were preheated to 80° C. for 15 minutes; then Novozyme 435 (33.4 mg) was added in one portion and the vial returned to heating. Reaction progress was monitored by removing aliquots of the reaction mixture and dissolving in 600 μL of CDCl₃. The catalyst was removed from the dissolved aliquot via syringe filtration. Conversion was determined by 1H NMR. The polymer was isolated by dissolving the reaction mixture in DCM, removing the catalyst via filtration, then precipitating the polymer with methanol.

Large scale solvent-free copolymerization using Novozyme 435. A 100 mL round bottom flask was charged with EB (7.64 g, 0.028 mol), DO (6.4 g, 0.028 mmol), and an overhead mechanical stirrer was carefully loaded into the reaction. The contents of the round bottom flask were preheated to 80° C. for 15 minutes; then Novozyme 435 (0.2 g) was added. The polymer was isolated by dissolving the reaction mixture in DCM, removing the catalyst via filtration, then precipitating the polymer with methanol. The polymer was then characterized using 1H NMR and GPC.

### (ii) Characterization of Poly(EB-co-DO) Copolymers

The structure of poly(EB-co-DO) catalyzed by Novozyme-435 was determined via ¹H and ¹³C NMR spectroscopy. ¹H NMR was used to determine the incorporation ratio of EB and DO in the copolymer. Percentage conversion and incorporation ratio was calculated using the 1H NMR signal of the R—COO—CH₂—CH₂—COO—R of PEB at 4.2 ppm and the —CH₂—CH₂—O—CH₂—COO— of PDO at 3.8 ppm (with reference to CDCl₃).

(iii) Implant Fabrication

For DEX-loaded implants, different ratios of poly(EB-co-DO) and DEX were dissolved in dichloromethane (DCM) and methanol, respectively. Both solutions were then sonicated and vortexed together to ensure a homogeneous distribution of drug throughout the polymer matrix and poured into a clean round bottom flask. For LNG-loaded or DTG-loaded implants, polymer and drug were both dissolved in DCM only. A rotary evaporator was used to evaporate the organic solvents over 1 hour. To form two-centimeter implants, the resulting polymer-drug pellets (110 mg) were loaded into a custom-machined Teflon mold and baked for 1 hour at 100° C. under argon protection and atmospheric pressure. The implants were compressed using a stainless-steel plunger immediately after baking and demolded the next day. For in vitro release and mechanical testing, the implants were cut in half to create two one-centimeter implants that were 2.4 mm in diameter and approximately 50 mg in total mass.

### (iv) Implant Characterization Via SEM

DEX-loaded poly(EB-co-DO) implants were evaluated under an ultra-high-resolution Hitachi scanning electron microscopy (SU7000). Implants were flash frozen in liquid nitrogen, broken in half using tweezers, and cross sectioned using a razor blade to about 1 mm thickness. Samples were placed on a stub using carbon tape, with razor blade edge facing down. Samples were coated with gold to a thickness of 7 nm using a high-resolution sputter coater (Cressington, 208 HR) with rotary planetary tilt stage and thickness controller MTM-20. SEM images were taken at 2k to 20k magnification.

### (v) In Vitro Drug Release

Drug-loaded poly(EB-co-DO) implants (1 cm long, 2.4 mm diameter, 50 mg) were individually incubated in 1 mL of PBS solution (pH=7.4) containing 3% methyl-beta-cyclodextrin (M-β-CD) at 37° C. At selected time points, the buffer solution was completely replaced with fresh solution of the same composition. The collected buffer solutions, in which the implant had been continuously and completely bathed for a period of time, were flash frozen and lyophilized. High-performance liquid chromatography (HPLC) was then used to determine the representative drug loading of implants in each group.

To determine drug concentrations, one mL of acetonitrile (ACN) was added to DEX and LNG samples to dissolve the agent or one mL of 1:1 water:ACN was added to DTG samples. The samples were then centrifuged at 3000 rpm for 5 min to draw out any salts and M-β-CD. The supernatant was passed through 0.22 um syringe filters before being analyzed by HPLC for drug content.

An Agilent column (883995-906, ZORBAX StableBond 300 C8, 4.6×150 mm, 5 μm) and Shimadzu HPLC system (LC-2030C) with a UV-Vis detector was used for drug quantitation. For both DEX and LNG, Solvent A was 0.1% TFA in HPLC grade water, and solvent B was 0.1% TFA in HPLC grade ACN. Initial solvent B concentration was set at 5% and allowed to increase to 100% by the 5th minute. Flow was maintained for 5 min at this level before dropping to 5% after 12 min, which was maintained until the end of the run at 13 minutes. For analysis of DTG drug release, Solvent A was 0.1% TFA in HPLC grade water, and solvent B was 100% methanol. Initial solvent B concentration was set at 55% and increased to 60% for the 6-7th minute before being brought back down to 55% for the 7-10th minute. All three drugs were detected using a UV detector at a wavelength of 240 nm. Oven temperature was 30° C. for DEX and LNG, and 35° C. for DTG. A volume of 45 μl was injected for each sample run.

### (vi) Mathematical Diffusion Model for Prediction of Drug Release

Fick's second law of diffusion appropriately models drug transport across polymeric matrixes by proportionally relating the diffusion rate at time, t, to the concentration gradient of drug in the implant, ∂C/∂r. As shown in the equation below (1), a one-dimensional, radial release from our cylindrical implants was adopted, with a constant drug diffusion coefficient, D.

$$\frac{\partial C}{\partial t} = D \frac{1}{r} \frac{d}{dr} [ r \frac{\partial C}{\partial r} ] \qquad ( 1 )$$

The solution for the above partial differential equation requires specification of a set of initial conditions and boundary conditions. For the initial condition (t=0), a homogenous and uniform concentration of drug throughout the implant was adopted, C=C₀. A symmetry boundary condition is further applied at the center of the implant (r=0) as flux across the plane of symmetry is zero, ∂C/∂r=0. Lastly, a perfect-sink boundary condition is applied at the surface of implant (r=R), C=0. Solving Equation 1 via numerical integration in MATLAB reveals predicted drug concentration in the implant, C, as a function of time, t, and radius, r. The predicted drug concentration is then integrated across the radius of the implant to determine the mass of drug remaining within the implant at each time step. In vitro release, then, is determined from the mass of drug in the implant at each time point subtracted from initial mass of drug within the implant (Co*V). Lastly, the diffusion coefficient is determined by fitting the cumulative drug release profile to data from in vitro drug release experiments and minimizing root mean squared error (RMSE).

(vii) Mechanical Testing

The mechanical properties of the DEX loaded poly(EB-co-DO) implants were assessed via three-point bend tests and compression tests using an Instron 5960 Mechanical Testing Machine.

The three-point bend test. The three-point bend test involved placing individual implants horizontally over a metal grip with prongs separated by a length (L) of 5 mm. A 2 kN load cell moved downwards at a speed of 2 mm/min and exerted a force in between the two prongs until the implant fractured or a maximum displacement of 20 mm was reached. Force (F), displacement (D), flexural stress (σ_{f}), and flexural strain (ε_{f}) were recorded by the Instron. The flexural modulus is defined as E_{f}=σ_{f}/ε_{f} where stress and strain are defined as σ_{f}=FL/πr³ and ε_{f}=6Dd/L² respectively |10|. The length of the implant was 10 mm, the diameter (d) was 2.4 mm, and the radius (r) was 1.2 mm.

The compression test. The compression test was similarly performed using a 2 kN load cell travelling at a speed of 2 mm/min. Implants were placed on top of a stainless-steel plate and were compressed in the transverse direction until a maximum displacement of 10 mm was reached or when the implant became fractured or irreversibly deformed. Force (F), displacement (D), compression stress (σ_{c}), and compression strain (ϵ_{c}) were recorded to calculate the compressive modulus of elasticity as E_{c}=σ_{c}/ε_{c}. The compressive modulus can also be calculated as

$${E}_{c} = \frac{8 ( 1 - {μ}^{2} ) {Z}^{2} F}{π dL} ,$$

which includes the additional parameters: Poisson's ratio (μ), geometric constant (Z=radius of implant/half contact width in radial compression), implant diameter (d), and implant length (L) (W. K. Solomon, V. K. Jindal, Comparison of axial and radial compression tests for determining elasticity modulus of potatoes, Int J Food Prop 9 (4) (2006) 855-862).
(viii) Thermal Characterization of Polymers

Thermogravimetric analysis (TGA) experiments were conducted using a Shimadzu instrument. First, 10 mg of polymer was inserted into the sample chamber and the temperature was increased at 10° C./min from 25° C. to 600° C. The degradation profile was collected during scanning. For the differential scanning calorimetry (DSC) experiments, a TA instrument DSC (TA instruments-DSC250) was used (precalibrated to 10° C./min). Each experiment required 5 to 8 mg of polymer; two cycles were performed consecutively.

### (ix) Density Measurements

To determine density, polymer samples of ˜200 mg were first massed with a Mettler Toledo XS205 DU balance with a resolution of 0.01 mg. Samples were then placed into a 1 cm³ chamber within the Micromeritics Accupyc II 1340 pycnometer, with helium as the gas for each fictive temperature characterized. Four measurements were performed for each sample. The standard deviation for the volume measurements by helium pycnometry is 0.001 cm³.

### Results

### (i) Small-Scale Synthesis

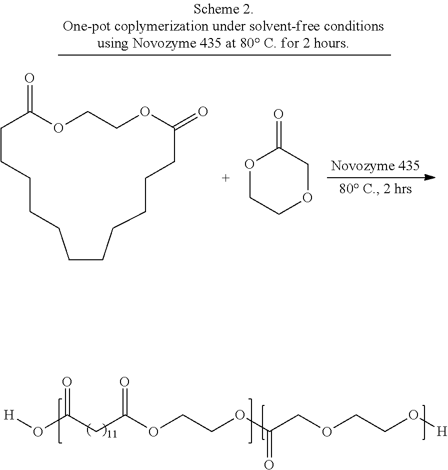

The one-pot copolymerization of EB and DO proceeds under solvent-free conditions catalyzed by N435 (Scheme 2). Conversion versus time data were acquired using a 1:1 feed ratio of EB and DO. At room temperature, DO has poor solubility in EB; thus, heating the monomers prior to addition of the catalyst proved to be important for early incorporation of DO into the polymer. As the polymerization progressed, the reaction mixture became too viscous to stir and solidified around 30 minutes or when EB reached 50-60% conversion; the solidification of the reaction mixture explains the premature plateau in conversion for DO. In addition, the monomer equilibrium concentration ([M]_{eq}) for DO is 2.5 M, which could also explain why DO did not reach full conversion (A. Heise, C. Duxbury, A. Palmans, Enzyme-Mediated Ring-Opening Polymerization, in: P. Dubois, O. Coulembier, J.-M. Raquez (Eds.), Handbook of Ring-Opening Polymerization 2009, pp. 379-397). The conversion versus time plot indicates the formation of a gradient block copolymer and reveals that after two hours, no further conversion of EB or DO is observed at 80° C.

Using the established time and temperature parameters, various monomer feed ratios and catalyst loadings were explored (Tables 2 and 3). The resulting polyesters possessed high weight-average molecular weights (M_{w}) and a broad scope of incorporation ratios. Changing the feed ratio resulted in polymers that had monomer incorporation ratios that suggest an increased catalyst selectivity for EB. A feed ratio of 50:50 EB:DO yields a polymer with the incorporation ratio of 58:42 and a M_{w} of 71k with a 16.7% catalyst loading (entry 5, Table 2). Broad molecular weight distributions (M_{w}/Mₙ>2) were observed, which are characteristic of solvent-free ROP due to the high viscosity of the macrolactone monomer that leads to an increase in undesirable chain transfer reactions. The ¹H and ¹³C NMR of poly(EB-co-DO) showed an incorporation ratio of 70:30.

Analogous to a degree of polymerization (DP) screen, reactions with various amounts of enzyme were studied under the solvent-free copolymerization conditions for a 50:50 feed ratio of EB:DO at 80° C. (Table 3). The 50:50 feed ratio achieved an incorporation ratio of approximately 60:40 with M_{w} values ranging from 49k to 72k g/mol. Cutting the amount of catalyst in half (Table 3; entries 1 and 2) resulted in the largest change in M_{w} (49k and 71k, respectively) and smaller differences in M_{w} were observed with 16.7-4.2 wt % catalyst loading (Table 3; entries 2-4). The DP or lengths of the polymer chains were not directly controlled by the amount of enzyme present. The active site of CALB includes a Ser-His-Asp catalytic triad that is essential in initiation and formation of the enzyme activated monomer (EAM). A lactone enters the active site and undergoes nucleophilic attack by the terminal alcohol in the Ser residue; this step is widely accepted as the rate determining step and forms the EAM. The amount of water in the active site plays a major role in polymer chain lengths and is responsible for hydrolysis and regeneration of the enzyme active site (EAS). Chain termination can occur through multiple pathways such as polycondensation, hydrolysis of the polymer chain end, or self-condensation to form cycles (A. E. Polloni, V. Chiaradia, E. M. Figura, J. De Paoli, D. de Oliveira, J. V. de Oliveira, P. H. H. de Araujo, C. Sayer, Polyesters from Macrolactones Using Commercial Lipase NS 88011 and Novozym 435 as Biocatalysts, Appl Biochem Biotech 184 (2) (2018) 659-672). The wide variety of termination mechanisms leads to the broad molecular weight distributions observed for this lipase-catalyzed copolymerization.

TABLE 2
Composition, molecular weight, and polydispersity for the lipase-
catalyzed copolymerization of EB and DO with varying feed ratios (reactions
were all conducted at 80° C.).

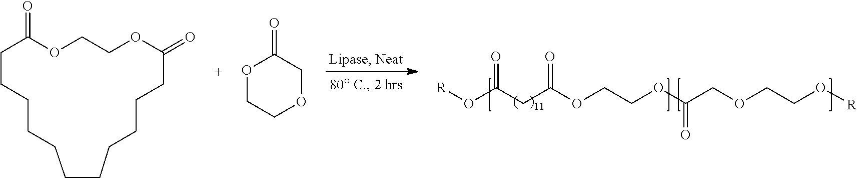

| | | | Amount of | | | |
|---|---|---|---|---|---|---|
| | | | enzyme (mg | | | |
| | EB:DO | | enzyme/ | | | |
| | Feed | Incorporation | mmol | Mₙ^{c} | M_{w}^{c} | |
| Entry | Ratio^{a} | Ratio^{b} | monomer) | (g/mol) | (g/mol) | PDI^{c} |
| 1 | 90:10 | 91:9 | 16.7 | 30,000 | 117,000 | 3.9 |
| 2 | 80:20 | 86:14 | 16.7 | 39,000 | 119,000 | 2.0 |
| 3 | 70:30 | 75:25 | 16.7 | 46,000 | 105,000 | 2.3 |
| 4 | 60:40 | 66:34 | 16.7 | 29,000 | 81,000 | 2.8 |
| 5 | 50:50 | 58:42 | 16.7 | 29,000 | 71,000 | 2.4 |
| 6 | 40:60 | 51:49 | 16.7 | 30,000 | 52,000 | 1.8 |

^{a}Total amount of monomer sums to 2 mmol
^{b}Incorporation ratio determined by ¹H-NMR.
^{c}M_{w}, Mₙ, and PDI determined by GPC in dichloromethane against polystyrene standards.

TABLE 3
Composition, molecular weight, and polydispersity
for the lipase-catalyzed copolymerizations
of EB and DO with varying catalyst loading

| | | | Amount of | | | |
|---|---|---|---|---|---|---|
| | | | enzyme (mg | | | |
| | EB:DO | | enzyme/ | | | |
| | Feed | Incorporation | mmol | Mₙ^{c} | M_{w}^{c} | |
| Entry | Ratio^{a} | Ratio^{b} | monomer) | (g/mol) | (g/mol) | PDI^{c} |
| 1 | 50:50 | 55:45 | 33.4 | 27,000 | 49,000 | 1.8 |
| 2 | 50:50 | 58:42 | 16.7 | 29,000 | 71,000 | 2.4 |
| 3 | 50:50 | 62:38 | 11.4 | 31,000 | 66,000 | 2.1 |
| 4 | 50:50 | 64:36 | 4.2 | 25,000 | 72,000 | 2.9 |

^{a}Total amount of monomer sums to 2 mmol
^{b}Incorporation ratio determined by ¹H-NMR.
^{c}M_{w}, Mₙ, and PDI determined by GPC in dichloromethane against polystyrene standards.

### (ii) Large-Scale Synthesis Reactions

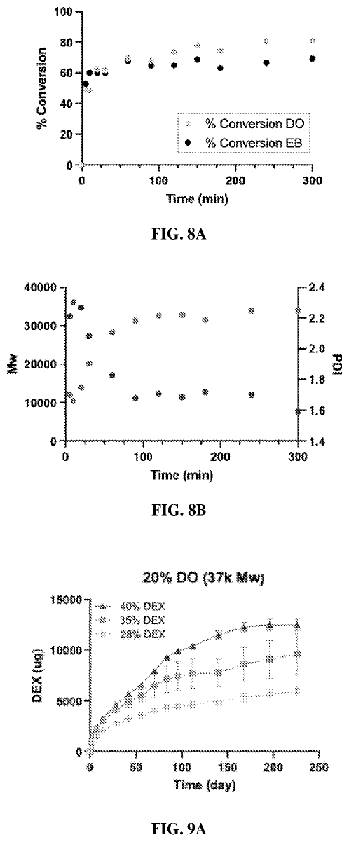

Having established suitable polymerization conditions for a small-scale, copolymerizations were carried out at a 25-g scale to explore the feasibility of large-scale production. Reactions were conducted in a nitrogen glove box equipped with a mechanical stirrer. One limitation of the large-scale synthesis was the hardening of the material and loss of stirring as the polymerization progressed when using a magnetic stir bar. Stirring with an overhead mechanical stirrer, however, allowed for continuous mixing of the contents of the flask throughout the two-hour polymerization. A conversion versus time plot for the large-scale reaction suggest a more random copolymerization (FIGS. 8A and 8B). The reaction reaches a plateau in conversion at 250 minutes, after which the molecular weight and PDI remains constant. It is also noted that the % conversion looked constant after 100 mins with slight variations. The large-scale reactions resulted in random copolymers with number average molecular weights (Mn) ranging from 30k to 55k and broad molecular weight distributions (Table 4). The material from these large-scale reactions was used for implant fabrication.

TABLE 4
Composition, molecular weight, and polydispersity for the
lipase-catalyzed copolymerization of EB and DO with varying
feed ratios for large-scale polymer synthesis.

| | | | Amount | | | |
|---|---|---|---|---|---|---|
| | EB:DO | Incorporation | of | | | |
| | Feed | Ratio | enzyme | Mₙ^{c} | M_{w}^{c} | |
| Polymer | Ratio^{a} | (EB:DO)^{b} | (mg) | (g/mol) | (g/mol) | PDI^{c} |
| 1 | 100:0 | 100:0 | 150 | 52,000 | 23,000 | 1.8 |
| 2 | 100:0 | 100:0 | 200 | 40,000 | 19,000 | 2.1 |
| 3 | 90:10 | 93:7 | 200 | 45,000 | 23,000 | 1.9 |
| 4 | 75:25 | 80:20 | 200 | 37,000 | 18,000 | 2.1 |
| 5 | 50:50 | 60:40 | 200 | 32,000 | 17,000 | 1.8 |

^{a}Total amount of monomer sums to 2 mmol
^{b}Incorporation ratio determined by ¹H-NMR.
^{c}M_{w}, Mₙ, and PDI determined by GPC in dichloromethane against polystyrene standards.

(iii) Implant Fabrication, In Vitro Release, and Polymer Degradability

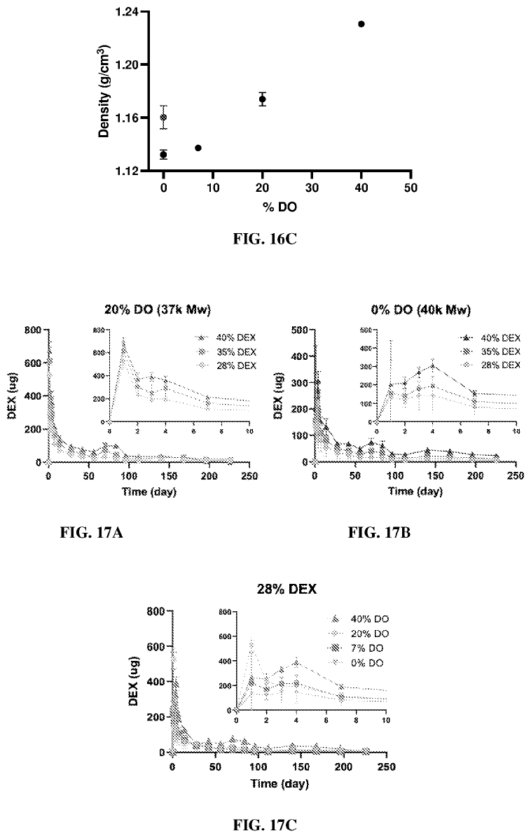

A series of poly(EB-co-DO) copolymers—with varying DO contents and number average molecular weights (Mₙ) ranging from 30k to 55k—were characterized and used for the fabrication of blank or drug-loaded implants (FIGS. 16A, 16B, and 16C). When characterized by thermogravimetric analysis (TGA), polymers with higher DO content degraded faster, with an onset of degradation at 170° C. for a copolymer with 40% DO and 290° C. for 20% DO. The DSC results showed a nonlinear decrease in the melting point Tₘ with increasing percentage of DO in the polymer backbone. Multiple melting peaks are seen in the DSC curves, suggesting that these polymers have a large number of crystalline populations that melt at different temperatures. Polymers of EB with M_{w}<90 kg/mol have been shown to be more heterogeneous (J. Fernandez, et al. J Mech Behav Biomed Mater 64 (2016) 209-219). Furthermore, pycnometry revealed an increase in polymer density with increasing percentage of DO (FIG. 16C).

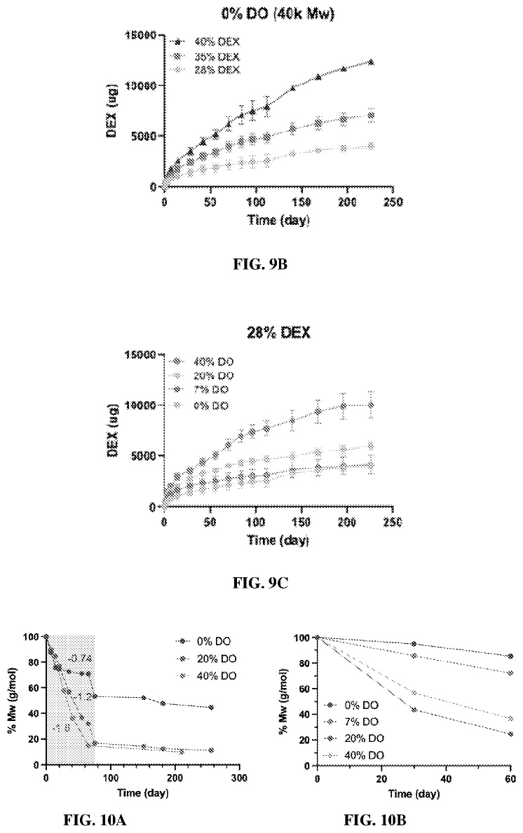

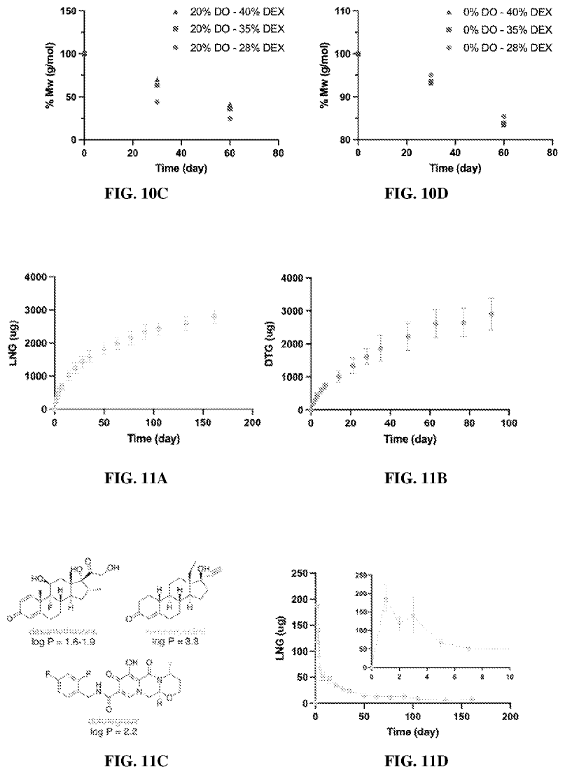

The poly(EB-co-DO) copolymers were used to produce 1-cm unloaded and drug-loaded implants according to an established melt-molding technique (W. Saltzman, et al, Biodegradable Contraceptive Implants, 2020). Implants loaded with 28%, 35%, and 40% drug possessed a theoretical loading of 14 mg, 18 mg, and 20 mg, respectively. DEX-loaded implants were fabricated and characterized for polymer degradation, in vitro drug release, and overall internal morphology. DEX release was dependent on both drug loading and DO content of the polymer. Higher DEX loading led to increased cumulative and daily drug release rates (FIGS. 9A-9C, FIGS. 17A-17C). The first five days of the daily release profile also revealed a burst release, likely due to rapid release of DEX near the implant surface. In the higher loading implants, the burst release was as much as three times higher than daily release at later times (FIGS. 17A-17C). Higher DO content in the polymer also resulted in faster drug release rate and faster polymer degradation (FIG. 9C and FIGS. 10A-10D). The faster release in higher DO content materials may be a consequence of increased number of ester linkages, which arise due to the increased number of repeating units of DO at a given Mw. Increasing the number of ester linkages would thus result in faster polymer degradation via hydrolysis. Increased DO content in the polymer also results in increased hydrophilicity, which could also enhance the adsorption of water, therefore increasing the rate of hydrolysis (FIG. 10A). The loading of DEX in the implants did not significantly influence the rate of polymer degradation (FIGS. 10B and 10D).

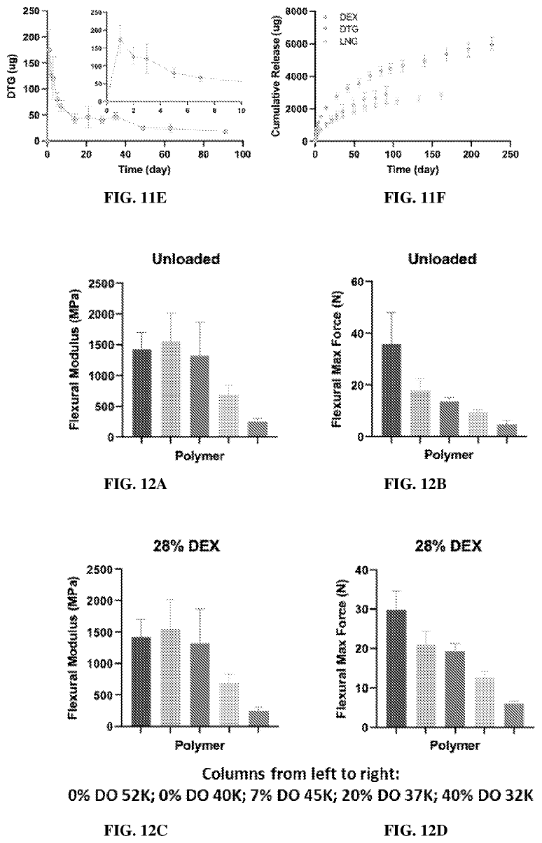

Implants were produced with two alternate agents incorporated: LNG and DTG. Twenty-eight percent LNG-loaded or DTG-loaded implants were fabricated with P4 (37k M_{w}, 20% DO). In vitro release profiles reveal a slower cumulative release for both LNG and DTG compared to DEX from comparable implants as well as a three-fold reduction in burst release rates, despite the same theoretical drug loading (FIGS. 11A-11F). These differences in release are likely a consequence of the greater hydrophilicity of DEX relative to LNG and DTG as reflected in their log P values: DEX (log P=1.6-1.9)>DTG (log P=2.2)>LNG (log P=3.3) (FIGS. 11C-11F).

A mathematical model was used to better understand drug release profiles, to predict the in vitro release kinetics of DEX from poly(EB-co-DO) implants over time periods beyond our experimental measurements, and to reveal the mechanism of controlled release in these implants. SEM images—as well as our experience of the handling implants after in vitro and in vivo exposure to release media-suggest that the implants remain mechanically strong throughout months of DEX release. Therefore, a stable continuous polymer phase and a homogeneous distribution of drug dissolved in the polymer matrix was adopted; thus DEX release was modeled assuming Fickian diffusion with an effective diffusion coefficient representing DEX diffusion through the polymer matrix (Saltzman W M, Drug Delivery: Engineering principles for drug therapy, Oxford University Press (2001)). Drug release from DEX-loaded implants were measured in vitro for 226 days; the models indicated that these implant will continue to release at a constant rate for at least another 275 days. Similar modeling of LNG and DTG release suggest that drug release for these drugs are comparable during the first 100 days but will likely deviate as time progresses. The model suggests that by 500 days less than half of the drug theoretically incorporated into each implant (14 mg) will be released. However, these models do not account for polymer degradation and erosion of the implant matrix: the model is only valid so long as the implant is intact and Fickian diffusion persists. By day 226 of in vitro release, it was noticed that 5 of the 36 DEX-loaded implants had started to physically erode, exposing millimeter-sized pores, with no clear trend with respect to drug loading or DO content.

The effective diffusion coefficients describing DEX release from poly(EB-co-DO) implants with varying drug loadings and DO content ranged from 1.1×10⁻⁸ to 9.0×10⁻⁹ cm²/sec (Table 5).

TABLE 5
Parameters resulting from mathematical diffusion
model for in vitro release of DEX.

| | | | | Diffusion | |
|---|---|---|---|---|---|
| | Mn | DO | % | Coefficient | RMSE |
| Polymer | (g/mol) | incorporation | Loading | (cm²/sec) | (ug) |
| P1 | 52,000 | 0% | 28 | 5.9 × 10⁻⁹ | 162.9 |
| P2 | 40,000 | 0% | 28 | 1.3 × 10⁻⁹ | 110.6 |
| P2 | | | 35 | 2.7 × 10⁻⁹ | 172.0 |
| P2 | | | 40 | 6.3 × 10⁻⁹ | 669.4 |
| P3 | 45,000 | 7% | 28 | 1.8 × 10⁻⁹ | 233.7 |
| P4 | 37,000 | 20% | 28 | 3.7 × 10⁻⁹ | 247.8 |
| P4 | | | 35 | 6.5 × 10⁻⁹ | 304.9 |
| P4 | | | 40 | 9.0 × 10⁻⁹ | 515.7 |
| P5 | 32,000 | 40% | 28 | 1.1 × 10⁻⁸ | 318.0 |

The diffusion coefficients for DTG and LNG in these implants are similar in order of magnitude (Table 6).

TABLE 6
Parameters resulting from mathematical diffusion
model for in vitro release of DEX, DTG, and LNG.

| | | Diffusion | |
|---|---|---|---|
| | | Coefficient | RMSE |
| Drug | % Loading | (cm²/sec) | (ug) |
| DEX | 28 | 3.7 × 10⁻⁹ | 247.8 |
| DTG | 28 | 1.7 × 10⁻⁹ | 112.8 |
| LNG | 28 | 1.0 × 10⁻⁹ | 98.7 |

Literature values for the diffusion coefficient of free DEX in in tissues has been found to be substantially higher (2.0−6.8×10⁻⁶ cm²/sec depending on the experimental conditions) (Y. Moussy, et al., Biotechnol Progr 22 (6) (2006) 1715-1719; L. Hersh, Mathematical techniques for the estimation of the diffusion coefficient and elimination constant of agents in subcutaneous tissue, Physics, University of South Florida, 2007, p. 81). In water at 37° C., radiolabeled DEX in saline was found to have a diffusion coefficient of 5.2×10⁻⁶ cm²/sec, whereas in porous ethylene-vinyl acetate copolymer (EVAc) implants with 35% DEX loading, the diffusion coefficient was four orders of magnitudes lower at 2.0×10⁻¹⁰ cm²/sec (W. M. Saltzman, et al, Chem Eng Sci 46 (10) (1991) 2429-2444). Results also indicate that the diffusion coefficient of DEX increases with increasing DO content, following an exponential relationship over the measured range of DO content.

### (iv) Mechanical Testing

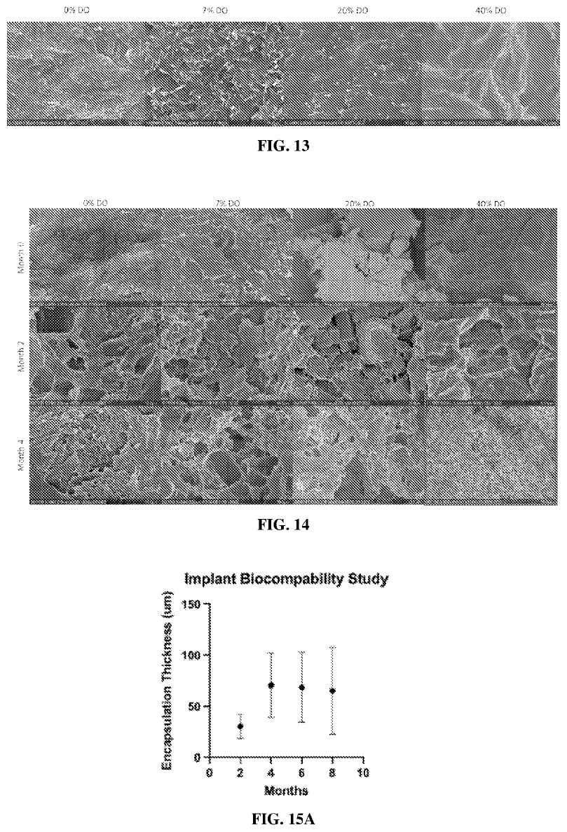

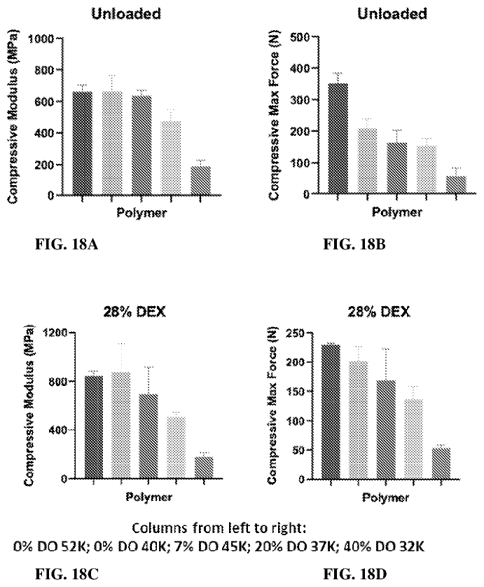

Mechanical testing of DEX-loaded implants was performed to evaluate their suitability as an implanted medical device. Results from three-point bend tests and compression tests revealed variation in mechanical properties with implant drug loading and polymer composition (FIGS. 12A-12D). For both tests, the modulus and maximum force to fracture were not statistically different between unloaded and DEX-loaded implants (FIGS. 13A-13D and FIGS. 18A-18D). For each drug loading, implants with a greater percentage of DO monomer were mechanically weaker, with reduced flexural and compressive modulus and reduced flexural and compressive maximum force before fracture (FIGS. 12A-12D and FIGS. 18A-18D). This trend was also observed qualitatively in the polymers prior to implant fabrication, as the 40% DO polymer was noticeably softer and gummier compared to the 0% DO polymer during routine handling.

During compressive testing on 0% and 20% DO implants, higher drug loading increased the compressive modulus but had no statistically significant effect on the compressive maximum force before failure. For three-point testing on 20% DO implants, increasing drug loading had no statistically significant effect on flexural modulus or flexural maximum force before failure. On the other hand, for 0% DO implants, increasing drug loading decreased both the flexural modulus and flexural maximum force before failure. The addition of drug to these implants effects mechanical strength in ways that are not yet predictable.

To examine mechanical strength after a period of degradation, implants from each group were sacrificed at Day 56 and Day 112 of in vitro release to determine flexural modulus and maximum force before facture. As expected, the flexural strength generally decreased over time, a feature likely attributed to increased porosity in implant microstructure as the polymer degrades and drug is released.

The mechanical strength of these implants is relevant as, in a clinical application, the implants must withstand mechanical forces from within the user's tissue on a continual basis. Although the main advantage of biodegradable contraceptive implants compared to commercial implants is the convenience of not needing an excision procedure, implants must remain structurally intact throughout use and they must be mechanically strong enough to survive a removal procedure, if needed during the use period. In clinical trials, the tensile integrity of implants is assessed by comparing implant breaking rates to that of other contraceptive implants. For instance, researchers noted a significantly higher breakage rate of LNG-releasing Sino-implants (II) compared to Jadelle® implants (16.3% vs 3.1%) during removal procedures (M. J. Steiner, et al., Randomized trial to evaluate contraceptive efficacy, safety and acceptability of a two-rod contraceptive implant over 4 years in the Dominican Republic, Contracept X 1 (2019) 100006). Determining the ease of implant removal from mice models is an obvious next step to assess implant tensile strength in vivo.

### (v) SEM Imaging

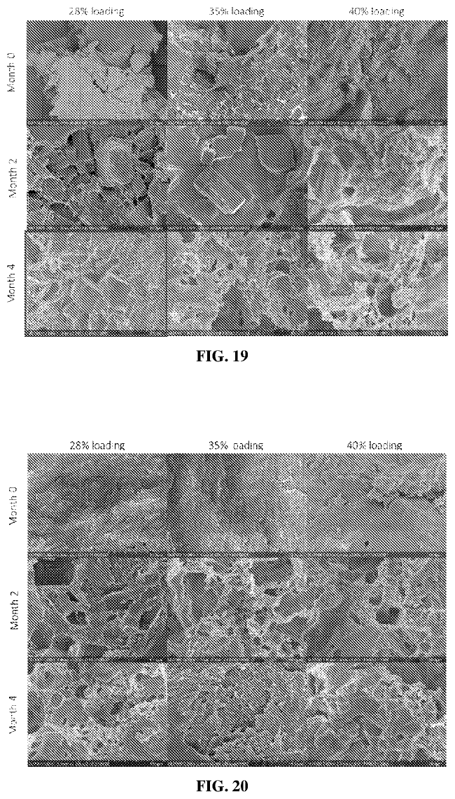

To better understand the effect on drug loading and DO content on internal microstructure of the implant during various stages of drug release, SEM images of cross sections of unloaded and DEX-loaded implants were examined. Among unloaded poly(EB-co-DO) implants, cross-sections of implants with 0, 7 or 20% DO appeared brittle and firm, as illustrated by the sharp ridges of the image. On the other hand, cross sections of the 40% DO implant appears softer in texture, as noted by the rounded edges (FIG. 13). These images corroborate the mechanical testing results that revealed greater compressive and flexural strength among implants with lower DO content. Cross-sections of DEX-loaded poly(EB-co-DO) implants reveal crystalline drug structures, identified by their distinct, cuboid-shaped protrusions. Compared to month 0, SEM images of cross-sections at months 2 and 4 reveal greater porosity in implants with 0-40% DO, irrespective of drug loading (FIG. 14, FIG. 19, and FIG. 20).

### (vi) Biocompatibility Study

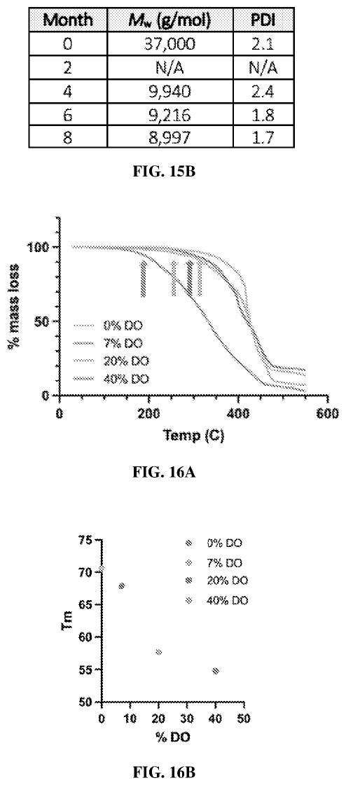

To examine tissue responses and in vivo degradation of these implants, unloaded 20% DO implants (P4) were used and examined histological sections and polymer properties over a period of 8 months after subcutaneous implantation. Implants of ˜10 to 15 mg were implanted in the subdermal layer of 16 mice. Three implants were extracted every two months; half of the implants were used for histological examination while the other half were used to characterize the polymer using GPC and NMR (FIGS. 15A and 15B). Histology results demonstrate an increase in encapsulation thickness to ˜75 um during the first four months, after which the encapsulation thickness remained constant (FIG. 15A). This encapsulation layer is much less thick than described in comparable experiments with PLGA and PLA implants: Previous studies with PLGA implants in mice showed an encapsulation thickness of greater than 100 μm within 7 to 60 days of implantation. Another study with PLA implants shows an encapsulation thickness of ˜300 μm. GPC results indicate that the Mw of the polymer decreased from 37 kg/mol to 10 kg/mol within a period of 4 months and then remained constant from month 4 to month 8 (FIG. 15B). This decrease in Mw (73%) during the first four months is slightly less than observed in vitro degradation, where there was an 84% decrease in Mw after four months of incubation in PBS buffer at 37° C. However, these results may not be comparable given that the starting Mw of the in vitro degradation study was slightly lower, at 29 kg/mol. This trend can likely be attributed to the highly degradable PDO polymeric segments of the copolymer. In terms of structural integrity of the implants, the implants appeared intact at 8 months and were firm enough to be picked up by tweezers without damage to the implant.

In sum, copolymerization reactions were conducted with EB and DO, resulting in reproducible production of poly(EB-co-DO) by enzyme-catalyzed ROP. These reactions were scaled up to produce an array of polymers with varying DO contents. These copolymers were used for the fabrication of rod-shaped biodegradable implants, which were successfully loaded with either DEX, LNG, and DTG. Drug release rates and implant mechanical properties depended on monomer content, polymer molecular weight, and drug loading, demonstrating that these implants can be tuned for desired properties of release and degradation. The poly(EB-co-DO) implants are well tolerated after subcutaneous implantation in mice, demonstrating their suitability for safe use for long-term drug release.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A non-injectable biodegradable controlled agent release implant for implantation in tissue comprising:
(A) a copolymer (i) poly(ω-pentadecalactone-co-p-dioxanone) having a general structure according to Formula (I) 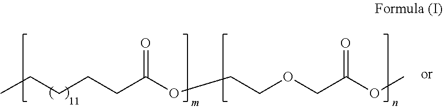
(ii) poly(ethylene brassylate-co-dioxanone) having a general structure according to Formula (Ia) or Formula (Ib): 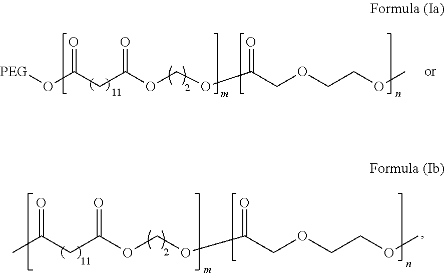
wherein in Formula (I), Formula (Ia), and Formula (Ib) n and m are independently integer values of less than or equal to about 1500,
and
(B) an effective amount of one or more antiviral agents to achieve a desired pharmaceutical dosage over a period of one to twenty-four months.

2. The implant of claim 1, wherein the poly(ω-pentadecalactone-co-p-dioxanone) or poly(ethylene brassylate-co-dioxanone) has a dioxanone (DO) mol % content in the range of about 20 to about 60%.

3. A biodegradable polymeric microparticulate formulation delivering an effective amount of two or more anti-viral agents for a period of at least two months.

4. The microparticulate formulation of claim 3 comprising a polymer selected from the group consisting of polyesters, poly(caprolactone), polycarbonates, polyamides, polyanhydrides, polyamino acids, polyortho esters, polyacetals, polycyanoacrylates, degradable polyurethanes, blends and copolymers thereof.

5. The implant of claim 1, wherein the one or more anti-viral agents are selected from the group consisting of anti-HIV, anti-influenza, anti-Ebola, and anti-coronavirus.

6. The implant of claim 1, wherein the one or more anti-viral agents are selected from the group consisting of Nucleoside reverse transcriptase inhibitors (NRTIs), Non-nucleoside reverse transcriptase inhibitors (NNRTIs), Protease inhibitors (PIs), Chemokine receptor antagonist (CCR5 antagonist), Fusion inhibitor (FI), Entry inhibitor, Integrase inhibitors (INSTIs), Complete Regimen Combination ARTs, Nucleoside/nucleotide reverse transcriptase inhibitors (NRTIs), Non-nucleoside Reverse Transcriptase Inhibitors (NNRTIs), Protease Inhibitors, Integrase Strand-Transfer Inhibitors, Fusion Inhibitors, Chemokine Receptor Antagonists, CD4 Post-attachment Inhibitors, gp120 Attachment Inhibitors, Pharmacokinetic Enhancers, combination of NRT1 and NNRT1 Compounds, and polymeric implants of a NNRT1 and NRT1.

7. The implant of claim 1, wherein the one or more anti-viral agents are selected from the group consisting of the NRT1, EFdA, and a NNRT1, Compound I, a catechol diether, the compounds having the following structures: 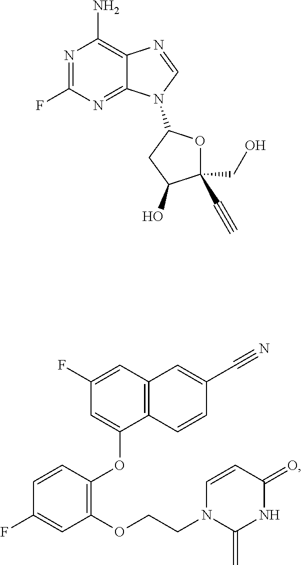
and active salts or metabolites thereof.

8. The implant of claim 1, wherein;
(i) the implant provides controlled sustained release for a period of at least twenty-four months,
(ii) the implant is suitable for subcutaneous or intramuscular implantation, or
(iii) the copolymer degrades over a period of up to about 24 months.

9. (canceled)

10. (canceled)

11. The implant of claim 1 in the form of a rod, cylinder, bead, film, or disk.

12. The implant of claim 1, having a percent antiviral agent loading from about 1% to about 80%, from about 1% to about 50%, from about 1% to about 40% by weight, from about 1% to about 20% by weight, or from about 1% to about 10% by weight, or a weight ratio of copolymer to one or more agents of about 3:1, 2.5:1, 2:1, 1:1.

13. The implant of claim 1, further comprising:
(i) additional therapeutic, prophylactic or diagnostic agents, or
(ii) a coating comprising a polyester copolymer.

14. (canceled)

15. The implant or formulation of claim 13, wherein the coating is free from the therapeutic.

16. The implant of claim 1, wherein the implant further comprises a core that is free from therapeutic.

17. A method of administering the implant of claim 1, comprising implanting the implant subcutaneously or intramuscularly in a subject in need thereof.

18. The formulation of claim 3 wherein the two or more anti-viral agents are selected from the group consisting of anti-HIV, anti-influenza, anti-Ebola, and anti-coronavirus.

19. The formulation of claim 3 wherein the two or more anti-viral agents are selected from the group consisting of Nucleoside reverse transcriptase inhibitors (NRTIs), Non-nucleoside reverse transcriptase inhibitors (NNRTIs), Protease inhibitors (PIs), Chemokine receptor antagonist (CCR5 antagonist), Fusion inhibitor (FI), Entry inhibitor, Integrase inhibitors (INSTIs), Complete Regimen Combination ARTs, Nucleoside/nucleotide reverse transcriptase inhibitors (NRTIs), Non-nucleoside Reverse Transcriptase Inhibitors (NNRTIs), Protease Inhibitors, Integrase Strand-Transfer Inhibitors, Fusion Inhibitors, Chemokine Receptor Antagonists, CD4 Post-attachment Inhibitors, gp120 Attachment Inhibitors, Pharmacokinetic Enhancers, combination of NRT1 and NNRT1 Compounds, and polymeric implants of a NNRT1 and NRT1.

20. The formulation of claim 3 wherein the two or more anti-viral agents are selected from the group consisting of the NRT1, EFdA, and a NNRT1, Compound I, a catechol diether, the compounds having the following structures: 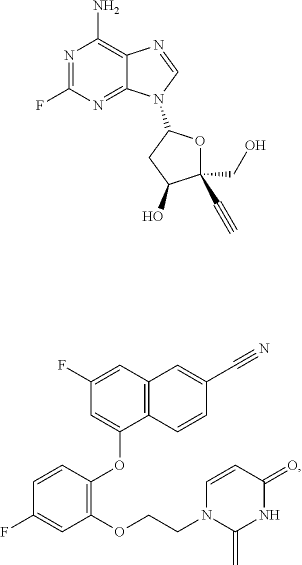
and active salts or metabolites thereof.

21. The formulation of claim 3:
(i) wherein the formulation provides controlled sustained release for a period of at least twenty-four months,
(ii) wherein the formulation is suitable for subcutaneous or intramuscular implantation,
(iii) wherein the microparticulate formulation comprises a copolymer that degrades over a period of up to about 24 months, or
(iv) having a percent antiviral agent loading from about 1% to about 80%, from about 1% to about 50%, from about 1% to about 40% by weight, from about 1% to about 20% by weight, or from about 1% to about 10% by weight, or a weight ratio of copolymer to one or more agents of about 3:1, 2.5:1, 2:1, 1:1.

23. The formulation of claim 3 further comprising:
(i) additional therapeutic, prophylactic or diagnostic agents, or
(ii) a coating comprising a polyester copolymer.

24. A method of administering the formulation of claim 3, comprising implanting the implant or particles subcutaneously or intramuscularly in a subject in need thereof.
